(19) United States
(12) Patent Application Publication
(10) Pub. No.: US 2022/0177471 A1
(43) Pub. Date: Jun. 9, 2022

(54) N-1 BRANCHED ALKYL SUBSTITUTED IMIDAZO[4,5-C]QUINOLINE COMPOUNDS, COMPOSITIONS, AND METHODS

(71) Applicant: 3M INNOVATIVE PROPERTIES COMPANY, St. Paul, MN (US)

(72) Inventors: George W. Griesgraber, Eagan, MN (US); Michael J. Rice, Woodbury, MN (US); Hannah C. Cohen, St. Paul, MN (US); Devon M. Hunerdosse, St. Paul, MN (US); Adam D. Miller, Vadnais Heights, MN (US); Joshua R. Wurst, North St. Paul, MN (US)

(21) Appl. No.: 17/594,415

(22) PCT Filed: May 27, 2020
(86) PCT No.: PCT/IB2020/055034

§ 371 Date: Oct. 15, 2021

Related U.S. Application Data
(60) Provisional application No. 62/858,009, filed on Jun. 6, 2019.

(51) Int. Cl. C07D 471/04

(52) U.S. Cl. CPC C07D 471/04

(57) ABSTRACT

Imidazo[4,5-c]quinoline compounds having a substituent that is attached at the N-1 position by a branched group, single enantiomers of the compounds, pharmaceutical compositions containing the compounds, and methods of making the compounds are disclosed. Methods of use of the compounds as immune response modifiers, for inducing (or inhibiting) cytokine biosynthesis in humans and animals, and in the treatment of diseases including infectious and neoplastic diseases are also disclosed.

### BACKGROUND

Some drug compounds act by stimulating certain key aspects of the immune system, as well as by suppressing certain other aspects (e.g., U.S. Pat. No. 6,039,969 (Tomai et al.) and U.S. Pat. No. 6,200,592 (Tomai et al.)). These compounds are sometimes referred to as immune response modifiers (IRMs). Some IRM compounds are useful for treating viral diseases, neoplasias, and T_{H}2-mediated diseases. Some IRM compounds are useful as vaccine adjuvants.

IRM compounds have been reported based on the following bicyclic and tricyclic ring systems: 1H-imidazo[4,5-c]quinolin-4-amines (e.g., U.S. Pat. No. 4,689,338 (Gerster)); 1H-imidazo[4,5-c]pyridin-4-amines (e.g., U.S. Pat. No. 5,446,153 (Lindstrom et al.)); 1H-imidazo[4,5-c][1,5]naphthyidin-4-amines (e.g., U.S. Pat. No. 6,194,425 (Gerster et al.)); thiazolo[4,5-c]quinolone-4-amines and oxazolo[4,5-c]quinolone-4-amines (e.g., U.S. Pat. No. 6,110,929 (Gerster et al.)); 6,7,8,9-1H-tetrahydro-1H-imidazo[4,5-c]quinolin-4-amines (e.g., U.S. Pat. No. 5,352,784 (Nikolaides et al.)); 2H-pyrazolo[3,4-c]quinolone-4-amines (e.g., U.S. Pat. No. 7,544,697 (Hays et al.)); and N-1 and 2-substituted 1H-imidazo[4,5-c]quinolin-4-amines (e.g., U.S. Pat. No. 6,331,539 (Crooks et al.), U.S. Pat. No. 6,451,810 (Coleman et al.), U.S. Pat. No. 6,664,264 (Dellaria et al.), U.S. Pat. No. 8,691,837 (Krepski et al.), U.S. Pat. No. 8,088,790 (Kshirsagar et al.), U.S. Pat. No. 8,673,932 (Kshirsagar et al.), U.S. Pat. No. 8,697,873 (Krepski et al.), and U.S. Pat. No. 7,915,281 (Krepski et al.)).

### SUMMARY

New compounds, salts thereof, and compositions including such compounds and salts that can be useful, for example, in inducing cytokine biosynthesis in humans and animals are disclosed. Such compounds are of the following Formula (I):

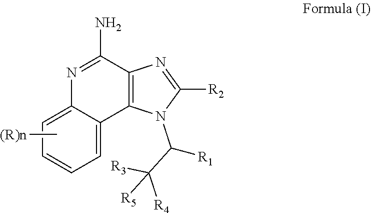

wherein:

n is an integer of 0 or 1;

R is selected from the group consisting of halogen, hydroxy, alkyl, alkoxy, and —C(O)—O-alkyl;

R₁ is a C₁₋₆ alkyl;

R₂ is selected from the group consisting of hydrogen, methyl, ethyl, n-propyl, n-butyl, —CH₂OCH₃, —CH₂OCH₂CH₃, and —CH₂CH₂OCH₃;

R₅ is selected from the group consisting of —H, —CH₃, —F, and —OH; and

R₃ is a C₁₋₄alkyl, R₄ is a C₁₋₄alkyl, or R₃ and R₄ are combined to form a ring of 3-7 carbon atoms, optionally having one oxygen atom in the ring, provided that R₅ is not —OH.

The compounds of Formula (I), and salts thereof, have a chiral center in the branched group off N-1. Thus, the compounds of Formula (I), and salts thereof, can be resolved, and/or synthesized using well-known techniques and chiral starting materials, into compounds of Formulas (II) and (III), and salts thereof:

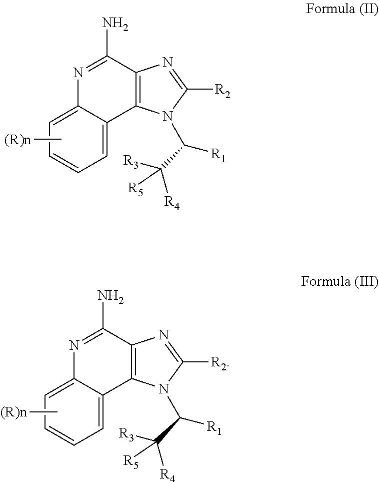

Compounds of Formula (I), particularly those of Formula (II), and salts thereof, such as pharmaceutically acceptable salts, of these compounds can be used as immune response modifiers due to their ability to induce cytokine biosynthesis (e.g., induce the synthesis of at least one cytokine) and otherwise modulate the immune response when administered to humans or animals. Compounds of Formula (I), particularly those of Formula (II), and salts thereof, can therefore be used in the treatment of a variety of conditions such as viral diseases and tumors that are responsive to such changes in the immune response. Compounds of Formula (I), particularly those of Formula (II), and salts thereof, can also be used as vaccine adjuvants when administered in combination with a vaccine.

Herein, when embodiments of Formulas (I), (II), and (III) are described, it is generally assumed that such statements refer to the compounds as well as the salts thereof.

Pharmaceutical compositions containing an effective amount of a compound (or salts thereof including pharmaceutically acceptable salts thereof) of Formula (I), such as a compound of Formula (II), Formula (III), or a combination thereof, are disclosed.

Also disclosed are methods of inducing cytokine biosynthesis in a human or animal, treating an infectious disease in a human or animal, and treating a neoplastic disease in a human or animal by administering to the human or animal a compound of Formula (I), particularly a compound of Formula (II), and pharmaceutically acceptable salts thereof. Also disclosed are methods of inhibiting cytokine biosynthesis in a human or animal using a compound of Formula (I), particularly a compound of Formula (III), and pharmaceutically acceptable salts thereof.

The term “alkyl” refers to a monovalent group that is a radical of an alkane and includes straight-chain, branched, cyclic, and bicyclic alkyl groups, and combinations thereof. Unless otherwise indicated, the alkyl groups typically contain from 1 to 20 carbon atoms. In some embodiments, the alkyl groups contain 1 to 12 carbon atoms, 1 to 10 carbon atoms, 1 to 9 carbon atoms, 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “alkyl” groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkylene” refers to a divalent group that is a radical of an alkane and includes groups that are linear, branched, cyclic, bicyclic, or a combination thereof. Unless otherwise indicated, the alkylene group typically has 1 to 20 carbon atoms. In some embodiments, the alkylene group has 1 to 12 carbon atoms, 1 to 10 carbon atoms, 1 to 9 carbon atoms, 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “alkylene” groups include methylene, ethylene, propylene, 1,4-butylene, 1,4-cyclohexylene, and 1,4-cyclohexyldimethylene.

The term “alkoxy” refers to a monovalent group having an oxy group bonded directly to an alkyl group.

The term “C_{x-y}alkyl” and “C_{x-y}alkoxy” are inclusive of straight chain groups, branched chain groups, cyclic groups, and combinations thereof that have X to Y carbon atoms. For example, a “C₁₋₅alkyl” includes alkyl groups of 1 carbon, 2 carbons, 3 carbons, 4 carbons, or 5 carbons. Some examples of “C₁₋₅alkyl” include methyl, ethyl, n-propyl, isopropyl, n-butyl, sec-butyl, isobutyl, isomeric pentyls, cyclopropyl, cyclopentyl, and —CH₂-cyclopropyl.

The “salt” of a compound includes pharmaceutically acceptable salts, such as those described in Berge, Stephen M., “Pharmaceutical Salts,” Journal of Pharmaceutical Sciences, 1977, 66, pages 1-19. For example, salts can be prepared by reacting a free base compound (that is, one not in a salt form) with an inorganic or organic acid such as, for example, hydrochloric acid, sulfuric acid, hydrobromic acid, methane sulfonic acid, ethane sulfonic acid, malic acid, maleic acid, acetic acid, trifluoroacetic acid, para-toluenesulfonic acid, salicylic acid, succinic acid, tartaric acid, citric acid, pamoic acid, xinafoic acid, oxalic acid, and the like. Typical pharmaceutically acceptable salts include hydrochloride and dihydrochloride.

As used herein, “pharmaceutically acceptable carriers” include those carriers that can deliver therapeutically or prophylactically effective amounts of one or more of the compounds or salts of the disclosure to a subject by a chosen route of administration, are generally tolerated by the subject, and have an acceptable toxicity profile (preferably minimal to no toxicity at an administered dose). Some suitable pharmaceutically acceptable carriers are described in Remington's Pharmaceutical Sciences, 18^{th} Edition (1990), Mack Publishing Co. and can be readily selected by one of ordinary skill in the art.

“Effective amount” (including “therapeutically effective amount” and “prophylactically effective amount”) are defined as an amount of compound or salt sufficient to induce a therapeutic or prophylactic effect, such as cytokine induction, immunomodulation, antitumor activity, and/or antiviral activity. Depending on the disease or condition, the desired cytokine profile, and/or the acceptable level of side effects, the effective amount may vary. For example, a small amount of a very active compound or salt, or a large amount of a compound or salt of low activity, may be used to avoid undesirable side effects.

“Treat” and “treatment” as well as variations thereof refer to reducing, limiting progression, ameliorating, preventing, or resolving to any extent the symptoms or signs related to a condition.

“Ameliorate” and “ameliorating” refers to any reduction in the extent, severity, frequency, and/or likelihood of a symptom or clinical characteristic of a particular disease or condition.

“Antigen” refers to any substance that can be bound by an antibody in a manner that is immunospecific to some degree.

Herein, the term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof).

The words “preferred” and “preferably” refer to embodiments of the disclosure that may afford certain benefits, under certain circumstances. However, other claims may also be preferred, under the same or other circumstances. Furthermore, the recitation of one or more preferred claims does not imply that other claims are not useful, and is not intended to exclude other claims from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein, the terms “ambient temperature” or “room temperature” refers to a temperature of 20° C. to 25° C. or 22° C. to 25° C.

The term “in the range” or “within a range” (and similar statements) includes the endpoints of the stated range.

Groupings of alternative elements or embodiments disclosed herein are not to be construed as limitations. Each group member may be referred to and claimed individually or in any combination with other members of the group or other elements found therein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

When a group is present more than once in a formula described herein, each group is “independently” selected, whether specifically stated or not. For example, when more than one R group is present in a formula, each R group is independently selected.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the invention. Thus, the appearances of such phrases in various places throughout this specification are not necessarily referring to the same embodiment of the invention. Furthermore, the particular features, configurations, compositions, or characteristics may be combined in any suitable manner in one or more embodiments.

The above summary of the present disclosure is not intended to describe each disclosed embodiment or every implementation of the present invention. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### DETAILED DESCRIPTION OF ILLUSTRATIVE EMBODIMENTS

This disclosure provides compounds (and salts thereof) of the following Formula (I):

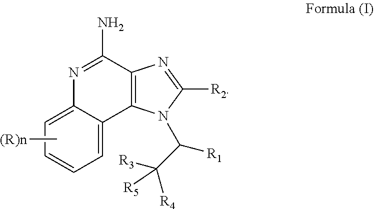

The compounds of Formula (I), and salts thereof, have a chiral center in the branched group off N-1. Thus, the compounds of Formula (I), and salts thereof, can be resolved, and/or synthesized using well-known techniques and chiral starting materials, into compounds of Formulas (II) and (III), and salts thereof:

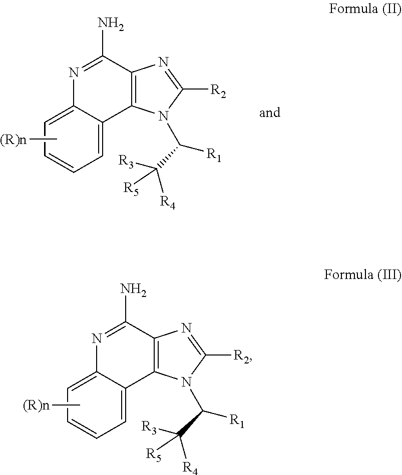

wherein:

n is an integer of 0 or 1;

R is selected from the group consisting of halogen, hydroxy, alkyl, alkoxy, and —C(O)—O-alkyl;

R₁ is a C₁₋₆alkyl;

R₂ is selected from the group consisting of hydrogen, methyl, ethyl, n-propyl, n-butyl, —CH₂OCH₃, —CH₂OCH₂CH₃, and —CH₂CH₂OCH₃;

R₅ is selected from the group consisting of —H, —CH₃, —F, and —OH; and

R₃ is a C₁₋₄alkyl, R₄ is a C₁₋₄alkyl, or R₃ and R₄ are combined to form a ring of 3-7 carbon atoms, optionally having one oxygen atom in the ring, provided that R₅ is not —OH.

Depending on the disease or condition, the desired cytokine profile, and/or the acceptable level of side effects, a compound of Formula (I), or salt thereof, may be more desirable than another compound of Formula (I), or salt thereof. Generally, a more active compound or salt of Formula (I) would be desirable for use in treating a viral disease, for example, whereas a less active compound of Formula (I), or salt thereof, may be used in certain situations, for example, to avoid undesirable side effects and/or for treating sensitive areas (e.g., mucous membranes).

Compounds of Formula (I) or salts thereof that are inactive toward cytokine production may be suitable in the treatment, e.g., of autoimmune conditions as a result of inhibiting cytokine biosynthesis. Examples of such compounds include those of Formula III, including compounds of Example 2, Example 4, Example 6, Example 10, Example 12, Example 14, Example 16, Example 18, and Example 21.

In some embodiments of Formulas (I), (II), and (III), R is selected from the group consisting of halogen, hydroxy, —C₁₋₁₂alkyl, —C₁₋₁₂alkoxy, and —C(O)—O—C₁₋₁₀alkyl. In some embodiments of Formulas (I), (II), and (III), R is selected from the group consisting of halogen, hydroxy, —C₁₋₇alkyl, —C₁₋₇alkoxy, and —C(O)—O—C₁₋₅alkyl. In some embodiments of Formulas (I), (II), and (III), R is selected from the group consisting of hydroxy, F, and Cl. In some embodiments of Formulas (I), (II), and (III), R is selected from the group consisting of F and Cl.

In some embodiments of Formulas (I), (II), and (III), n is 0.

In some embodiments of Formulas (I), (II), and (III), R₁ is a C₁₋₄alkyl. In some embodiments of Formulas (I), (II), and (III), R₁ is a C₃₋₆alkyl. In some embodiments of Formulas (I), (II), and (III), R₁ is a C₃₋₄alkyl.

In some embodiments of Formulas (I), (II), and (III), R₂ is hydrogen. In some embodiments of Formulas (I), (II), and (III), R₂ is selected from the group consisting of methyl, ethyl, n-propyl, and n-butyl. In some embodiments of Formulas (I), (II), and (III), R₂ is selected from the group consisting of hydrogen, methyl, and ethyl. In some embodiments of Formulas (I), (II), and (III), R₂ is selected from the group consisting of —CH₂OCH₃, —CH₂OCH₂CH₃, and —CH₂CH₂OCH₃.

In some embodiments of Formulas (I), (II), and (III), R₃ is a C₁₋₄alkyl. In some embodiments of Formulas (I), (II), and (III), R₃ is methyl or ethyl. In some embodiments of Formulas (I), (II), and (III), R₃ is methyl. In some embodiments of Formulas (I), (II), and (III), R₃ is ethyl.

In some embodiments of Formulas (I), (II), and (III), R₄ is a C₁₋₄alkyl. In some embodiments of Formulas (I), (II), and (III), R₄ is methyl or ethyl. In some embodiments of Formulas (I), (II), and (III), R₄ is methyl. In some embodiments of Formulas (I), (II), and (III), R₄ is ethyl.

In some embodiments of Formulas (I), (II), and (III), R₃ and R₄ are each methyl. In some embodiments of Formulas (I), (II), and (III), R₃ and R₄ are each ethyl.

In some embodiments of Formulas (I), (II), and (III), R₅ is —H, —CH₃, —F, or —OH. In some embodiments of Formulas (I), (II), and (III), R₅ is not —OH (i.e., R₅ is —H, —CH₃, or —F). In some embodiments of Formulas (I), (II), and (III), R₅ is —H, —F, or —OH. In some embodiments of Formulas (I), (II), and (III), R₅ is —H. In some embodiments of Formulas (I), (II), and (III), R₅ is —CH₃. In some embodiments of Formulas (I), (II), and (III), R₅ is —OH. In some embodiments of Formulas (I), (II), and (III), R₅ is —F.

In some embodiments of Formulas (I), (II), and (III), provided that R₅ is not —OH (i.e., R₅ is selected from the group consisting of —H, —CH₃, and —F), R₃ and R₄ are combined to form a ring of 3-7 carbon atoms, optionally having one oxygen atom in the ring. In some embodiments of Formulas (I), (II), and (III), provided that R₅ is not —OH (i.e., R₅ is selected from the group consisting of —H, —CH₃, and —F), R₃ and R₄ are combined to form a ring of 3-7 carbon atoms. In some embodiments of Formulas (I), (II), and (III), provided that R₅ is not —OH (i.e., R₅ is selected from the group consisting of —H, —CH₃, and —F), R₃ and R₄ are combined to form a ring of 3-7 carbon atoms having one oxygen atom in the ring. In some of these embodiments of Formulas (I), (II), and (III), R₅ is —H, and R₃ and R₄ are combined to form a ring of 3-7 carbon atoms, optionally having one oxygen atom in the ring. In some of these embodiments of Formulas (I), (II), and (III), R₅ is —CH₃, and R₃ and R₄ are combined to form a ring of 3-7 carbon atoms, optionally having one oxygen atom in the ring. In some of these embodiments of Formulas (I), (II), and (III), R₅ is —F, and R₃ and R₄ are combined to form a ring of 3-7 carbon atoms, optionally having one oxygen atom in the ring.

In some embodiments of Formulas (I), (II), and (III): R₁ is a C₁₋₆alkyl (preferably, R₁ is selected from the group consisting of —CH₃, —CH₂CH₃, —CH₂CH₂CH₃, —CH₂CH₂CH₂CH₃, and —CH₂CH(CH₃)₂); R₂ is selected from the group consisting of hydrogen, methyl, and ethyl (preferably, R₂ is hydrogen); R₃ is a C₁₋₄alkyl; R₄ is a C₁₋₄alkyl; R₅ is selected from the group consisting of —H, —CH₃, —F, and —OH; and n is 0. In some embodiments of such compounds, R₅ is —H. In some embodiments of such compounds, R₅ is —F. In some embodiments of such compounds, R₅ is —OH.

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂; R₂ is selected from the group consisting of hydrogen, methyl, and ethyl; R₃ is methyl or ethyl; R₄ is methyl or ethyl; R₅ is selected from the group consisting of —H, —CH₃, —F, and —OH; and n is 0.

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is selected from the group consisting of —H, —CH₃, —F, and —OH; and n is 0.

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂, R₂ is hydrogen; R₃ is methyl or ethyl; R₄ is methyl or ethyl; R₅ is hydrogen; and n is 0.

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl; R₄ is methyl; R₅ is hydrogen; and n is 0. Examples of such compounds include: 1-[(1R)-1,2-dimethylpropyl]imidazo[4,5-c]quinolin-4-amine (Example 1); 1-[(1S)-1,2-dimethylpropyl]imidazo[4,5-c]quinolin-4-amine (Example 2); and 1-[(1R)-1-isopropylpentyl]imidazo[4,5-c]quinolin-4-amine (Example 7).

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is —CH₃; and n is 0. Examples of such compounds include:
1-[(1R)-1,2,2-trimethylpropyl]imidazo[4,5-c]quinolin-4-amine (Example 3);
1-[(1S)-1,2,2-trimethylpropyl]imidazo[4,5-c]quinolin-4-amine (Example 4); and
1-[(1R)-1-tert-butylpentyl]imidazo[4,5-c]quinolin-4-amine (Example 8).

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is —F; and n is 0. Examples of such compounds include:
1-[(1R)-2-fluoro-1,2-dimethyl-propyl]imidazo[4,5-c]quinolin-4-amine (Example 20); and
1-[(1S)-2-fluoro-1,2-dimethyl-propyl]imidazo[4,5-c]quinolin-4-amine (Example 21).

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is —OH; and n is 0. Examples of such compounds include:
(3R)-3-(4-aminoimidazo[4,5-c]quinolin-1-yl)-2-methyl-butan-2-ol (Example 9);
(3S)-3-(4-aminoimidazo[4,5-c]quinolin-1-yl)-2-methyl-butan-2-ol (Example 10);
(3R)-3-(4-aminoimidazo[4,5-c]quinolin-1-yl)-2-methyl-pentan-2-ol (Example 11);
(3S)-3-(4-aminoimidazo[4,5-c]quinolin-1-yl)-2-methyl-pentan-2-ol (Example 12);
(3R)-3-(4-aminoimidazo[4,5-c]quinolin-1-yl)-2-methyl-hexan-2-ol (Example 13);
(3S)-3-(4-aminoimidazo[4,5-c]quinolin-1-yl)-2-methyl-hexan-2-ol (Example 14);
(3R)-3-(4-aminoimidazo[4,5-c]quinolin-1-yl)-2-methyl-heptan-2-ol (Example 15);
(3S)-3-(4-aminoimidazo[4,5-c]quinolin-1-yl)-2-methyl-heptan-2-ol (Example 16);
(3R)-3-(4-aminoimidazo[4,5-c]quinolin-1-yl)-2,5-dimethyl-hexan-2-ol (Example 17);
(3S)-3-(4-aminoimidazo[4,5-c]quinolin-1-yl)-2,5-dimethyl-hexan-2-ol (Example 18); and
(2R)-2-(4-aminoimidazo[4,5-c]quinolin-1-yl)-3-ethyl-pentan-3-ol (Example 19).

In some embodiments of Formulas (I), (II), and (III): R₁ C₁₋₆alkyl; R₂ is selected from the group consisting of hydrogen, methyl, and ethyl; R₃ and R₄ are combined to form a ring of 3-7 carbon atoms, optionally having one oxygen atom in the ring; R₅ is selected from the group consisting of —H, —CH₃, and —F (in some embodiment, R₅ is —H); and n is 0.

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ and R₄ are combined to form a ring of 3-7 carbon atoms, optionally having one oxygen atom in the ring; R₅ is selected from the group consisting of —H, —CH₃, and —F (in some embodiment, R₅ is —H); and n is 0.

In some embodiments of Formulas (I), (II), and (III): R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ and R₄ are combined to form a ring of 3-7 carbon atoms; R₅ is —H; and n is 0. Examples of such compounds include:
1-[(1R)-1-cyclohexylethyl]imidazo[4,5-c]quinolin-4-amine (Example 5); and 1-[(1S)-1-cyclohexylethyl]imidazo[4,5-c]quinolin-4-amine (Example 6).

In some embodiments of Formulas (I), (II), and (III), the compound is present in the form of a salt. The salt is typically a pharmaceutically acceptable salt. Most commonly the salt is a hydrochloride salt.

In some embodiments, mixtures of enantiomeric compounds, or salts thereof, of Formulas (II) and (III) are present.

In some embodiments, the compound of Formula (II), or salt thereof, has an enantiomeric purity of at least 80% enantiomeric excess (80% ee). The enantiomeric purity of a compound of Formula (II), or salt thereof, is relative to a compound of Formula (III), or salt thereof. In some embodiments, the compound of Formula (II), or salt thereof, has an enantiomeric purity of at least 90% enantiomeric excess (90% ee). In some embodiments, the compound of Formula (II), or salt thereof, has an enantiomeric purity of at least 95% enantiomeric excess (95% ee). In some embodiments, the compound of Formula (II), or salt thereof, has an enantiomeric purity of at least 97% enantiomeric excess (97% ee). In some embodiments, the compound of Formula (II), or salt thereof, has an enantiomeric purity of at least 98% enantiomeric excess (98% ee). In some embodiments, the compound of Formula (II), or salt thereof, has an enantiomeric purity of at least 99% enantiomeric excess (99% ee). In some embodiments, the compound of Formula (II), or salt thereof, has an enantiomeric purity of at least 99.5% enantiomeric excess (99.5% ee). In some embodiments, the compound of Formula (II), or salt thereof, has an enantiomeric purity of at least 99.8% enantiomeric excess (99.8% ee).

In some embodiments, the compound of Formula (III), or salt thereof, has an enantiomeric purity of at least 80% enantiomeric excess (80% ee). The enantiomeric purity of a compound of Formula (III), or salt thereof, is relative to a compound of Formula (II), or salt thereof. In some embodiments, the compound of Formula (III), or salt thereof, has an enantiomeric purity of at least 90% enantiomeric excess (90% ee). In some embodiments, the compound of Formula (III), or salt thereof, has an enantiomeric purity of at least 95% enantiomeric excess (95% ee). In some embodiments, the compound of Formula (III), or salt thereof, has an enantiomeric purity of at least 97% enantiomeric excess (97% ee). In some embodiments, the compound of Formula (III), or salt thereof, has an enantiomeric purity of at least 98% enantiomeric excess (98% ee). In some embodiments, the compound of Formula (III), or salt thereof, has an enantiomeric purity of at least 99% enantiomeric excess (99% ee). In some embodiments, the compound of Formula (III), or salt thereof, has an enantiomeric purity of at least 99.5% enantiomeric excess (99.5% ee). In some embodiments, the compound of Formula (III), or salt thereof, has an enantiomeric purity of at least 99.8% enantiomeric excess (99.8% ee).

Exemplary compounds of Formulas (I), (II), and (III) are presented in Tables 1-15. In the Tables 1-15, each row represents a specific compound with n, R₁, R₂, R₃, R₄, and R₅ defined.

TABLE 1

| n | R₁ | R₂ | R₃ | R₄ | R₅ |
|---|---|---|---|---|---|
| 0 | —CH₃ | —H | —CH₃ | —CH₃ | —H |
| 0 | —CH₂CH₃ | —H | —CH₃ | —CH₃ | —H |
| 0 | —CH₂CH₂CH₃ | —H | —CH₃ | —CH₃ | —H |
| 0 | —CH₂CH₂CH₂CH₃ | —H | —CH₃ | —CH₃ | —H |
| 0 | —CH₂CH(CH₃)₂ | —H | —CH₃ | —CH₃ | —H |
| 0 | —CH₂CH₂CH₂CH₂CH₃ | —H | —CH₃ | —CH₃ | —H |
| 0 | —CH₂CH₂CH₂CH₂CH₂CH₃ | —H | —CH₃ | —CH₃ | —H |

TABLE 2

| n | R₁ | R₂ | R₃ | R₄ | R₅ |
|---|---|---|---|---|---|
| 0 | —CH₃ | —H | —CH₃ | —CH₃ | —CH₃ |
| 0 | —CH₂CH₃ | —H | —CH₃ | —CH₃ | —CH₃ |
| 0 | —CH₂CH₂CH₃ | —H | —CH₃ | —CH₃ | —CH₃ |
| 0 | —CH₂CH₂CH₂CH₃ | —H | —CH₃ | —CH₃ | —CH₃ |
| 0 | —CH₂CH(CH₃)₂ | —H | —CH₃ | —CH₃ | —CH₃ |
| 0 | —CH₂CH₂CH₂CH₂CH₃ | —H | —CH₃ | —CH₃ | —CH₃ |
| 0 | —CH₂CH₂CH₂CH₂CH₂CH₃ | —H | —CH₃ | —CH₃ | —CH₃ |

TABLE 3

| n | R₁ | R₂ | R₃ | R₄ | R₅ |
|---|---|---|---|---|---|
| 0 | —CH₃ | —H | —CH₃ | —CH₃ | —OH |
| 0 | —CH₂CH₃ | —H | —CH₃ | —CH₃ | —OH |
| 0 | —CH₂CH₂CH₃ | —H | —CH₃ | —CH₃ | —OH |
| 0 | —CH₂CH₂CH₂CH₃ | —H | —CH₃ | —CH₃ | —OH |
| 0 | —CH₂CH(CH₃)₂ | —H | —CH₃ | —CH₃ | —OH |
| 0 | —CH₂CH₂CH₂CH₂CH₃ | —H | —CH₃ | —CH₃ | —OH |
| 0 | —CH₂CH₂CH₂CH₂CH₂CH₃ | —H | —CH₃ | —CH₃ | —OH |

TABLE 4

| n | R₁ | R₂ | R₃ | R₄ | R₅ |
|---|---|---|---|---|---|
| 0 | —CH₃ | —H | —CH₃ | —CH₃ | —F |
| 0 | —CH₂CH₃ | —H | —CH₃ | —CH₃ | —F |
| 0 | —CH₂CH₂CH₃ | —H | —CH₃ | —CH₃ | —F |
| 0 | —CH₂CH₂CH₂CH₃ | —H | —CH₃ | —CH₃ | —F |
| 0 | —CH₂CH(CH₃)₂ | —H | —CH₃ | —CH₃ | —F |
| 0 | —CH₂CH₂CH₂CH₂CH₃ | —H | —CH₃ | —CH₃ | —F |
| 0 | —CH₂CH₂CH₂CH₂CH₂CH₃ | —H | —CH₃ | —CH₃ | —F |

TABLE 5

| n | R₁ | R₂ | R₃ | R₄ | R₅ |
|---|---|---|---|---|---|
| 0 | —CH₃ | —H | —CH₃ | —CH₂CH₃ | —H |
| 0 | —CH₂CH₃ | —H | —CH₃ | —CH₂CH₃ | —H |
| 0 | —CH₂CH₂CH₃ | —H | —CH₃ | —CH₂CH₃ | —H |
| 0 | —CH₂CH₂CH₂CH₃ | —H | —CH₃ | —CH₂CH₃ | —H |
| 0 | —CH₂CH(CH₃)₂ | —H | —CH₃ | —CH₂CH₃ | —H |
| 0 | —CH₂CH₂CH₂CH₂CH₃ | —H | —CH₃ | —CH₂CH₃ | —H |
| 0 | —CH₂CH₂CH₂CH₂CH₂CH₃ | —H | —CH₃ | —CH₂CH₃ | —H |

TABLE 6

| n | R₁ | R₂ | R₃ | R₄ | R₅ |
|---|---|---|---|---|---|
| 0 | —CH₃ | —H | —CH₃ | —CH₂CH₃ | —CH₃ |
| 0 | —CH₂CH₃ | —H | —CH₃ | —CH₂CH₃ | —CH₃ |
| 0 | —CH₂CH₂CH₃ | —H | —CH₃ | —CH₂CH₃ | —CH₃ |
| 0 | —CH₂CH₂CH₂CH₃ | —H | —CH₃ | —CH₂CH₃ | —CH₃ |
| 0 | —CH₂CH(CH₃)₂ | —H | —CH₃ | —CH₂CH₃ | —CH₃ |
| 0 | —CH₂CH₂CH₂CH₂CH₃ | —H | —CH₃ | —CH₂CH₃ | —CH₃ |
| 0 | —CH₂CH₂CH₂CH₂CH₂CH₃ | —H | —CH₃ | —CH₂CH₃ | —CH₃ |

TABLE 7

| n | R₁ | R₂ | R₃ | R₄ | R₅ |
|---|---|---|---|---|---|
| 0 | —CH₃ | —H | —CH₃ | —CH₂CH₃ | —OH |
| 0 | —CH₂CH₃ | —H | —CH₃ | —CH₂CH₃ | —OH |
| 0 | —CH₂CH₂CH₃ | —H | —CH₃ | —CH₂CH₃ | —OH |
| 0 | —CH₂CH₂CH₂CH₃ | —H | —CH₃ | —CH₂CH₃ | —OH |
| 0 | —CH₂CH(CH₃)₂ | —H | —CH₃ | —CH₂CH₃ | —OH |
| 0 | —CH₂CH₂CH₂CH₂CH₃ | —H | —CH₃ | —CH₂CH₃ | —OH |
| 0 | —CH₂CH₂CH₂CH₂CH₂CH₃ | —H | —CH₃ | —CH₂CH₃ | —OH |

TABLE 8

| n | R₁ | R₂ | R₃ | R₄ | R₅ |
|---|---|---|---|---|---|
| 0 | —CH₃ | —H | —CH₃ | —CH₂CH₃ | —F |
| 0 | —CH₂CH₃ | —H | —CH₃ | —CH₂CH₃ | —F |
| 0 | —CH₂CH₂CH₃ | —H | —CH₃ | —CH₂CH₃ | —F |
| 0 | —CH₂CH₂CH₂CH₃ | —H | —CH₃ | —CH₂CH₃ | —F |
| 0 | —CH₂CH(CH₃)₂ | —H | —CH₃ | —CH₂CH₃ | —F |
| 0 | —CH₂CH₂CH₂CH₂CH₃ | —H | —CH₃ | —CH₂CH₃ | —F |
| 0 | —CH₂CH₂CH₂CH₂CH₂CH₃ | —H | —CH₃ | —CH₂CH₃ | —F |

TABLE 9

| n | R₁ | R₂ | R₃ | R₄ | R₅ |
|---|---|---|---|---|---|
| 0 | —CH₃ | —H | —CH₂CH₃ | —CH₂CH₃ | —H |
| 0 | —CH₂CH₃ | —H | —CH₂CH₃ | —CH₂CH₃ | —H |
| 0 | —CH₂CH₂CH₃ | —H | —CH₂CH₃ | —CH₂CH₃ | —H |
| 0 | —CH₂CH₂CH₂CH₃ | —H | —CH₂CH₃ | —CH₂CH₃ | —H |
| 0 | —CH₂CH(CH₃)₂ | —H | —CH₂CH₃ | —CH₂CH₃ | —H |
| 0 | —CH₂CH₂CH₂CH₂CH₃ | —H | —CH₂CH₃ | —CH₂CH₃ | —H |
| 0 | —CH₂CH₂CH₂CH₂CH₂CH₃ | —H | —CH₂CH₃ | —CH₂CH₃ | —H |

TABLE 10

| n | R₁ | R₂ | R₃ | R₄ | R₅ |
|---|---|---|---|---|---|
| 0 | —CH₃ | —H | —CH₂CH₃ | —CH₂CH₃ | —CH₃ |
| 0 | —CH₂CH₃ | —H | —CH₂CH₃ | —CH₂CH₃ | —CH₃ |
| 0 | —CH₂CH₂CH₃ | —H | —CH₂CH₃ | —CH₂CH₃ | —CH₃ |
| 0 | —CH₂CH₂CH₂CH₃ | —H | —CH₂CH₃ | —CH₂CH₃ | —CH₃ |
| 0 | —CH₂CH(CH₃)₂ | —H | —CH₂CH₃ | —CH₂CH₃ | —CH₃ |
| 0 | —CH₂CH₂CH₂CH₂CH₃ | —H | —CH₂CH₃ | —CH₂CH₃ | —CH₃ |
| 0 | —CH₂CH₂CH₂CH₂CH₂CH₃ | —H | —CH₂CH₃ | —CH₂CH₃ | —CH₃ |

TABLE 11

| n | R₁ | R₂ | R₃ | R₄ | R₅ |
|---|---|---|---|---|---|
| 0 | —CH₃ | —H | —CH₂CH₃ | —CH₂CH₃ | —OH |
| 0 | —CH₂CH₃ | —H | —CH₂CH₃ | —CH₂CH₃ | —OH |
| 0 | —CH₂CH₂CH₃ | —H | —CH₂CH₃ | —CH₂CH₃ | —OH |
| 0 | —CH₂CH₂CH₂CH₃ | —H | —CH₂CH₃ | —CH₂CH₃ | —OH |
| 0 | —CH₂CH(CH₃)₂ | —H | —CH₂CH₃ | —CH₂CH₃ | —OH |
| 0 | —CH₂CH₂CH₂CH₂CH₃ | —H | —CH₂CH₃ | —CH₂CH₃ | —OH |
| 0 | —CH₂CH₂CH₂CH₂CH₂CH₃ | —H | —CH₂CH₃ | —CH₂CH₃ | —OH |

TABLE 12

| n | R₁ | R₂ | R₃ | R₄ | R₅ |
|---|---|---|---|---|---|
| 0 | —CH₃ | —H | —CH₂CH₃ | —CH₂CH₃ | —F |
| 0 | —CH₂CH₃ | —H | —CH₂CH₃ | —CH₂CH₃ | —F |
| 0 | —CH₂CH₂CH₃ | —H | —CH₂CH₃ | —CH₂CH₃ | —F |
| 0 | —CH₂CH₂CH₂CH₃ | —H | —CH₂CH₃ | —CH₂CH₃ | —F |
| 0 | —CH₂CH(CH₃)₂ | —H | —CH₂CH₃ | —CH₂CH₃ | —F |
| 0 | —CH₂CH₂CH₂CH₂CH₃ | —H | —CH₂CH₃ | —CH₂CH₃ | —F |
| 0 | —CH₂CH₂CH₂CH₂CH₂CH₃ | —H | —CH₂CH₃ | —CH₂CH₃ | —F |

TABLE 13

| n | R₁ | R₂ | R₃-R₄ | R₅ |
|---|---|---|---|---|
| 0 | —CH₃ | —H | —CH₂CH₂CH₂CH₂— | —H |
| 0 | —CH₂CH₃ | —H | —CH₂CH₂CH₂CH₂— | —H |
| 0 | —CH₂CH₂CH₃ | —H | —CH₂CH₂CH₂CH₂— | —H |
| 0 | —CH₂CH₂CH₂CH₃ | —H | —CH₂CH₂CH₂CH₂— | —H |
| 0 | —CH₂CH(CH₃)₂ | —H | —CH₂CH₂CH₂CH₂— | —H |
| 0 | —CH₂CH₂CH₂CH₂CH₃ | —H | —CH₂CH₂CH₂CH₂— | —H |
| 0 | —CH₂CH₂CH₂CH₂CH₂CH₃ | —H | —CH₂CH₂CH₂CH₂— | —H |

TABLE 14

| n | R₁ | R₂ | R₃-R₄ | R₅ |
|---|---|---|---|---|
| 0 | —CH₃ | —H | —CH₂CH₂CH₂CH₂CH₂— | —H |
| 0 | —CH₂CH₃ | —H | —CH₂CH₂CH₂CH₂CH₂— | —H |
| 0 | —CH₂CH₂CH₃ | —H | —CH₂CH₂CH₂CH₂CH₂— | —H |
| 0 | —CH₂CH₂CH₂CH₃ | —H | —CH₂CH₂CH₂CH₂CH₂— | —H |
| 0 | —CH₂CH(CH₃)₂ | —H | —CH₂CH₂CH₂CH₂CH₂— | —H |
| 0 | —CH₂CH₂CH₂CH₂CH₃ | —H | —CH₂CH₂CH₂CH₂CH₂— | —H |
| 0 | —CH₂CH₂CH₂CH₂CH₂CH₃ | —H | —CH₂CH₂CH₂CH₂CH₂— | —H |

TABLE 15

| n | R₁ | R₂ | R₃-R₄ | R₅ |
|---|---|---|---|---|
| 0 | —CH₃ | —H | —CH₂CH₂OCH₂CH₂— | —H |
| 0 | —CH₂CH₃ | —H | —CH₂CH₂OCH₂CH₂— | —H |
| 0 | —CH₂CH₂CH₃ | —H | —CH₂CH₂OCH₂CH₂— | —H |
| 0 | —CH₂CH₂CH₂CH₃ | —H | —CH₂CH₂OCH₂CH₂— | —H |
| 0 | —CH₂CH(CH₃)₂ | —H | —CH₂CH₂OCH₂CH₂— | —H |
| 0 | —CH₂CH₂CH₂CH₂CH₃ | —H | —CH₂CH₂OCH₂CH₂— | —H |
| 0 | —CH₂CH₂CH₂CH₂CH₂CH₃ | —H | —CH₂CH₂OCH₂CH₂— | —H |

The disclosure provides a method of inducing cytokine biosynthesis in a human or animal by administering to the human or animal an effective amount of a compound or salt selected from the group consisting of any of the above embodiments of Formula (I), particularly embodiments of Formula (II).

The disclosure provides a method of inducing IFN-alpha biosynthesis in a human or animal by administering to the human or animal an effective amount of a compound or salt selected from any of the above embodiments of Formula (I), particularly embodiments of Formula (II).

The disclosure provides a method of inducing IFN-gamma biosynthesis in a human or animal by administering to the human or animal an effective amount of a compound or salt selected from any of the above embodiments of Formula (I), particularly embodiments of Formula (II).

The disclosure provides a method of inducing TNF-alpha biosynthesis in a human or animal by administering to the human or animal an effective amount of a compound or salt selected from any of the above embodiments of Formula (I), particularly embodiments of Formula (II).

The disclosure provides a method for treating an infectious disease (e.g., a viral, bacterial, fungal, or parasitic infection) in a human or animal by administering to the human or animal an effective amount of a compound or salt selected from any of the above embodiments of Formula (I), particularly embodiments of Formula (II).

The disclosure provides a method for treating a neoplastic disease in a human or animal by administering to the human or animal an effective amount of a compound or salt selected from any of the above embodiments of Formula (I), particularly embodiments of Formula (II).

The compounds, and salts thereof, of the disclosure may be synthesized by synthetic routes that include processes analogous to those well known in the chemical arts, particularly in light of the description contained herein. The starting materials are generally available from commercial sources such as the Sigma-Aldrich Company (St. Louis, Mo.) or are readily prepared using methods well known to those of ordinary skill in the art (e.g., prepared by methods generally described in Louis F. Fieser and Mary Fieser, Reagents for Organic Synthesis, v. 1-26, Wiley, New York; Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der Organischen Chemie, 4, Aufl. Ed. Springer-Verlag, Berlin, Germany, including supplements (also available via the Beilstein online database)).

Compounds of the disclosure can be prepared, for example, according to Reaction Schemes I, II, III and IV where R, R₁, R₂, R₃, R₄, R₅ and n are as described above. In Reaction Scheme I, a 4-chloro-3-nitroquinoline of Formula V is reacted in step (1) with an anime compound of Formula IV to provide a 3-nitroquinolin-4-amine of Formula VI. The reaction can be carried out by adding the amine of Formula IV to a solution of Formula V in a suitable solvent such as dichloromethane in the presence of a tertiary amine such as triethylamine. The 4-chloro-3-nitroquinoline compound of Formula V and substituted analogs are known compounds (see, for example, U.S. Pat. No. 3,700,674 (Diehl et al.), U.S. Pat. No. 5,389,640 (Gerster et al.), U.S. Pat. No. 6,110,929 (Gerster et al.), U.S. Pat. No. 7,923,560 (Wightman et al.), and references cited therein). In many cases, substituted analogs of Formula V (for example n=1 and R being a halogen, alkoxy or benzyloxy group) can be prepared starting with commercially available substituted anilines.

In step (2) of Reaction Scheme I, the nitro group of Formula VI can be reduced to an amino group. The reduction can be carried out in a pressure bottle using hydrogen, a catalytic amount of palladium or platinum on carbon, and a solvent such as methanol, acetonitrile, toluene, or combinations thereof. The reaction can be carried out with a Parr apparatus. Alternatively, the desired reduction can be accomplished using sodium dithionite and catalytic dioctyl viologen in a two phase dichloromethane-water solvent system. In step (3) of Reaction Scheme I, the resulting 3,4-diamine compound can be reacted with a carboxylic acid (R₂CO₂H) to provide a 1H-imidazo[4,5-c]quinoline of Formula VII. Suitable equivalents to carboxylic acids such as acyl chlorides, thioesters, and 1,1-dialkoxyalkyl alkanoates can also be used. The carboxylic acid or equivalent is selected so that it will provide the desired R₂ substituent in a compound of Formula VII. For example, triethylorthoformate will provide a compound where R₂ is hydrogen and trimethyl orthovalerate will provide a compound where R₂ is n-butyl. The reaction can be carried out without a solvent or with an inert solvent (for example ethyl acetate, n-propyl acetate or toluene). Optionally, a catalyst such as pyridine hydrochloride can be included.

In step (4) of Reaction Scheme I, the 1H-imidazo[4,5-c]quinoline of Formula VII can be oxidized to provide a 1H-imidazo[4,5-c]quinoline-5N-oxide using a conventional oxidizing agent capable of forming an N-oxide. Preferably, a solution of the compound of Formula VII in a suitable solvent such as chloroform or dichloromethane is reacted with 3-chloroperbenzoic acid (MCPBA) at ambient temperature.

In step (5) of Reaction Scheme I, the N-oxide compound can be aminated to provide a 1H-imidazo[4,5-c]quinoline-4-amine of Formula I. Step (5) involves reacting the N-oxide compound with an acylating agent and an aminating agent in an inert solvent such as dichloromethane or chloroform. Suitable acylating agents include alkyl- or arylsulfonyl chlorides such as benzenesulfonyl chloride, methanesulfonyl chloride, or para-toluenesulfonyl chloride. Ammonium hydroxide is a suitable aminating agent. The compound of Formula I can optionally be isolated as an organic or inorganic salt (for example as an HCl salt).

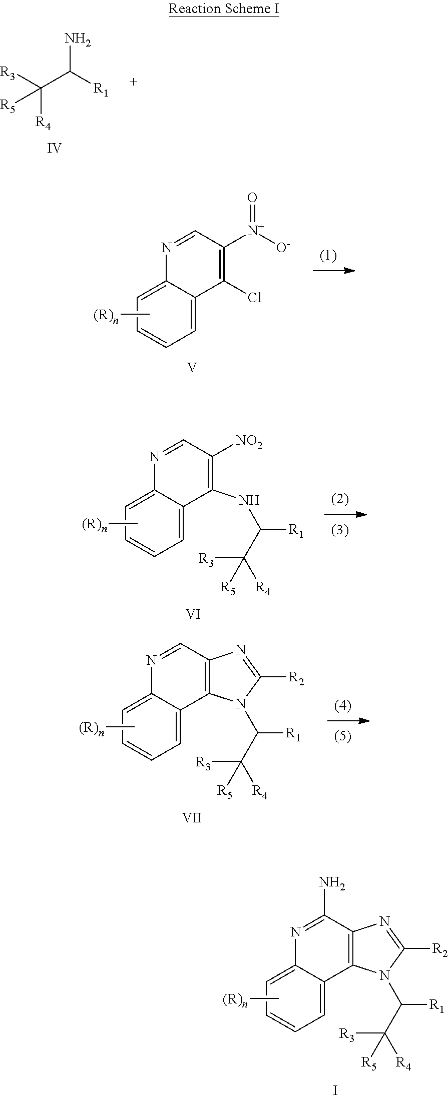

Many anime compounds of Formula IV in Scheme I are commercially available (for example 3-methylbutan-2-amine, 3,3-dimethylbutan-2-amine and 1-cyclohexylethanamine). Others can be prepared from commercially available alpha-amino carboxylic acids (for example alanine, 2-aminobutyric acid, 2-aminopentanoic acid, 2-aminohexanoic acid, and leucine). In step (6) of Reaction Scheme II an alpha-amino carboxylic acid of Formula VIII can be esterified by conventional methods such as reacting with thionyl chloride in an alcohol solvent (for example methanol or ethanol) to provide the amino ester as the hydrochloride salt. The esterification can also be achieved by reacting the alpha-amino carboxylic acid with a stoichiometric amount of a sulfonic acid (for example para-toluene sulfonic acid) in an alcohol solvent (for example methanol or ethanol) to provide the amino ester as the sulfonic acid salt. In step (7), the primary amine can be reacted with di-tert-butyl-dicarbonate [Boc₂O] and triethylamine to provide the Boc protected amine compound of Formula IX.

In step (8) of Reaction Scheme II, a Grignard reaction can be used to convert the ester substituent of Formula IX to the tertiary alcohol of Formula X. Examples of suitable Grignard reagents include methyl magnesium bromide, ethyl magnesium bromide, n-propyl magnesium chloride and the like. The Boc amino protecting group in the compound of Formula X can be removed in step (9) by reacting the compound of Formula X with hydrochloric acid in an alcohol solvent (for example methanol or ethanol) to provide the primary amine compound of Formula XI. It is often convenient to isolate the compound of Formula XI as a hydrochloride salt. The compound of Formula XI can be further reacted according to steps (1-5) described in Reaction Scheme I to provide compounds of Formula I where R₅ is —OH.

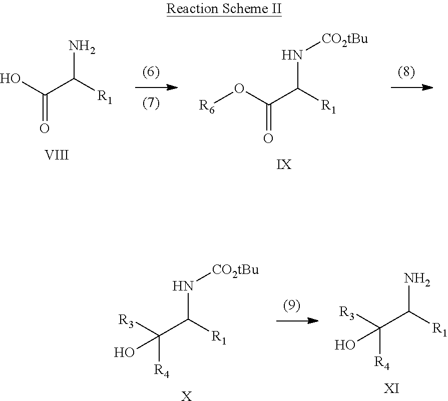

In Step 10 of Reaction Scheme III, the alcohol group of Formula X can be converted to a fluoride by treatment with a fluorinating agent such a diethylamino sulfur trifluoride in a suitable solvent such as methylene chloride to give a compound of Formula XII. The Boc amino protecting group in the compound of Formula XII can be removed in step (11) by reacting the compound of Formula XII with hydrochloric acid in an alcohol solvent (for example methanol or ethanol) to provide the primary amine compound of Formula XIII. It is often convenient to isolate the compound of Formula XIII as a hydrochloride salt. The compound of Formula XIII can be further reacted according to steps (1-5) described in Reaction Scheme I to provide compounds of Formula I where R₅ is —F.

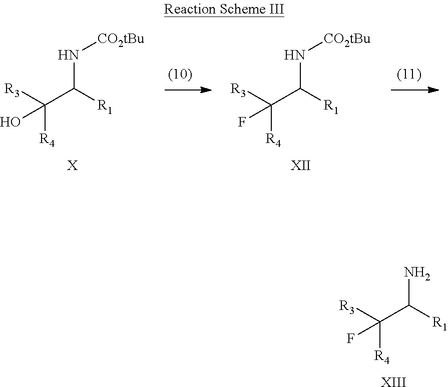

In step (12) of Reaction Scheme IV, an amino substituted carboxylic acid (for example tert-leucine) of Formula XIV can be reduced to an alcohol by reaction with iodine and sodium borohydride in an ether solvent. In step (13), the primary amine can be reacted with di-tert-butyl-dicarbonate [Boc₂O] and triethylamine to provide the Boc protected amine compound of Formula XV. In some cases, amino alcohols are commercially available (for example valinol) eliminating the need for step (12).

In step (14) of Reaction Scheme IV, the alcohol of Formula XV can be oxidized to an aldehyde by a variety of methods known to one skilled in the art. In particular, the method described by D. A. Six et al. (J. Med. Chem., 2007, 50, pages 4222-4235) using (2,2,6,6-tetramethylpiperidin-1-yl)oxyl (TEMPO) and sodium hypochlorite can be employed to oxidize Boc protected amino alcohols of formula XV to aldehydes of Formula XVI.

In step (15) of Reaction Scheme IV, the aldehyde of Formula XVI can be subjected to Wittig reaction conditions to provide the olefin compound of Formula XVII (where R₇ is —H or C₁₋₄ alkyl). In the Wittig reaction, alkyl triphenylphosphonium salts can be reacted with a base to form a phosphorus-carbon ylide. Examples of suitable alkyl triphenylphosphonium salts include methyl triphenylphosphonium bromide, ethyl triphenylphosphonium bromide, n-propyl triphenylphosphonium bromide and the like. Examples of suitable bases include sodium hydride, butyl lithium and potassium hexamethyldisilazide. The aldehyde of Formula XVI can then be reacted with the triphenylphosphonium ylide in a suitable solvent such as toluene to provide the olefin compound of Formula XVII. The obtained olefin is typically formed in the Z-configuration (as drawn), but in some instances can also be in the E-configuration.

In step (16) of Reaction Scheme IV, the olefin can be reduced to form a saturated alkyl group. The reduction can be carried out in a pressure bottle using hydrogen, a catalytic amount of palladium or platinum on carbon, and a solvent such as methanol, acetonitrile, toluene, or combinations thereof. The reaction can be carried out with a Parr apparatus. In step (17), the Boc amino protecting group in can be removed by reacting with hydrochloric acid in an alcohol solvent (for example methanol or ethanol) to provide the primary amine compound of Formula XVIII. It is often convenient to isolate the compound of Formula XVIII as a hydrochloride salt. The compound of Formula XVIII can be further reacted according to steps (1-5) described in Reaction Scheme I to provide compounds of Formula (I) where R₁ is C_{2-C6}alkyl.

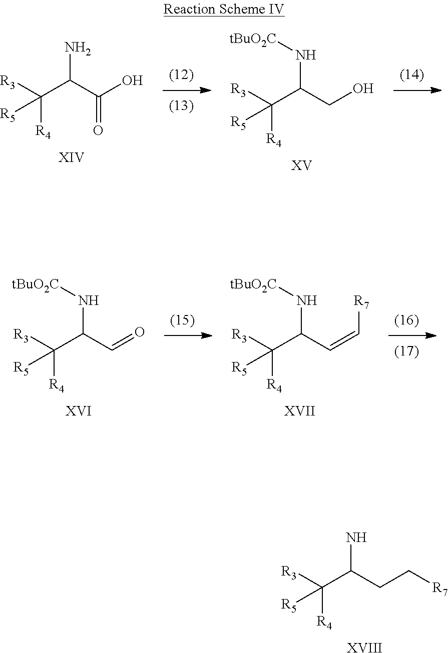

For Reaction Schemes I-IV, the compounds are drawn as racemic. It is understood that these reaction schemes can also be followed starting with compounds of high enantiomeric purity (for example a D or L amino acids) to prepare final compounds of the disclosure in high enantiomeric purity.

Alternatively, a racemic mixture of reactants or reactants of low enantiomeric purity (for example 10-70% enantiomeric excess) can be used with the final product isolated as the desired Formula (II) enantiomer using any suitable procedure for the resolution of a mixture of enantiomers. A well-known method for the resolution of a mixture of enantiomers is HPLC using a column with a chiral stationary phase (CSP). Another standard method for the resolution of a mixture of enantiomers involves reacting the mixture with an optically pure carboxylic acid to form diastereomeric salts that can be readily separated by for example recrystallization or chromatography methods. Regeneration of the free base completes the resolution process. Examples of resolving agents that are available in high enantiomeric purity include, but are not limited to, (+)-tartaric acid, (−)-mandelic acid, (−)-malic acid, (+)-camphor-10-sulfonic acid, and (+)-2,3-dibenzoyltartaric acid. If needed, different types of resolution steps can be combined and multiple resolution steps can be utilized to achieve the desired enantiomeric purity. The enantiomeric purity is represented as the percent enantiomeric excess (% ee). Methods for the resolution of isomers are described in the references: Y. Okamoto, Chemical Society Reviews, 2008, 37, pages 2593-2608; G. Gubitz, Biopharmaceutics and Drug Disposition, 2001, 22, pages 291-336; and S. Mane, Analytical Methods, 2016, 8, pages 7567-7586.

In the preparation of the compounds, or salts thereof, of the disclosure it is understood by one of ordinary skill in the art that it may be necessary to protect a particular functional group while reacting other functional groups of an intermediate compound. The need for such protection will vary depending on the nature of the particular functional group and the conditions of the particular reaction step. A review of reactions for protecting and deprotecting functional groups can be found in P. G. M. Wuts, Greene's Protective Groups in Organic Synthesis, John Wiley & Sons, New York, USA, 2014.

Conventional methods and techniques of separation and purification can be used to isolate the IRM compounds used in the compositions of the disclosure. Such techniques may include, for example, all types of chromatography (high performance liquid chromatography (HPLC), column chromatography using common absorbents such as silica gel, and thin layer chromatography), recrystallization, and differential (i.e., liquid-liquid) extraction techniques.

The enantiomeric excess of the compounds, or salts thereof, of the disclosure can be determined using standard analytical assays such as gas chromatography or HPLC with a column having a chiral stationary phase (CSP). Suitable columns with a CSP are available from Chiral Technologies, Inc., Westchester, Pa.

Enantiomeric excess (% ee) is calculated according to Equation 1.

$$enantiomeric excess ( \% ee ) = \frac{\begin{matrix}( mole \% of major enantiomer ) - \\ ( mol \% of minor enantiomer )\end{matrix}}{\begin{matrix}( mole \% of major enantiomer ) + \\ ( mole \% of minor enantiomer )\end{matrix}} \times 100. \qquad Equation 1$$

Enantiomeric excess (% ee) can be calculated from a chiral HPLC chromatogram by comparing the peak areas of the major enantiomer and minor enantiomer signals according to Equation 2.

$$enantiomeric excess ( \% ee ) = \frac{\begin{matrix}( peak area of major enantiomer ) - \\ ( peak area of minor enantiomer )\end{matrix}}{\begin{matrix}( peak area of major enantiomer ) + \\ ( peak area of minor enantiomer )\end{matrix}} \times 100. \qquad Equation 2$$

Prodrugs of the disclosed compounds can also be prepared by attaching to the compounds a functional group that can be cleaved under physiological conditions. Typically, a cleavable functional group will be cleaved in vivo by various mechanisms (such a through a chemical (e.g., hydrolysis) or enzymatic transformation) to yield a compound of the disclosure. A discussion of the use of prodrugs is provided by T. Higuchi and W. Stella. “Prodrugs as Novel Delivery Systems”, vol. 14 of the ACS Symposium Series, and in Bioreversible Carriers in Drug Design, ed. Edward B. Roche, American Pharmaceutical Association and Pergamon Press, 1987.

### Pharmaceutical Compositions and Biological Activity

Pharmaceutical compositions of the disclosure are also contemplated. Pharmaceutical compositions of the disclosure contain a therapeutically effective amount of a compound or salt of the disclosure (described herein) in combination with a pharmaceutically acceptable carrier.

Compounds of Formula (I), which may be compounds of Formula (II) and/or Formula (III), or salts thereof, may be provided in any pharmaceutical composition suitable for administration to a subject (human or animal) and may be present in the pharmaceutical composition in any suitable form (for example as a solution, a suspension, an emulsion, or any form of a mixture). The pharmaceutical composition may be formulated with any pharmaceutically acceptable excipient, carrier, or vehicle. In some embodiments, the pharmaceutically acceptable carrier comprises water (for example phosphate buffered saline or citrate buffered saline). In some embodiments, the pharmaceutically acceptable carrier comprises an oil (for example corn, sesame, cottonseed, soybean, or safflower oil). The pharmaceutical composition may further include one or more additives including suspending agents, surfactants, dispersing agents, and preservatives (such as an anti-oxidant).

In some embodiments of the pharmaceutical composition, the compounds of Formula (I), which may be compounds of Formula (II) and/or Formula (III), or salts thereof, can be incorporated in a homogeneously dispersed formulation. In some embodiments of the pharmaceutical composition, the compounds of Formula (I), which may be compounds of Formula (II) and/or Formula (III), or salts thereof, can be incorporated in an emulsified formulation. In some embodiments of the pharmaceutical composition, the compounds of Formula (I), which may be compounds of Formula (II) and/or Formula (III), or salts thereof, can be incorporated in an oil-in-water formulation. An oil-in-water formulation can comprise an oil component, an aqueous component, and one or more surfactants (for example, formulations comprising soybean oil, TWEEN 80, SPAN 85, and phosphate buffered saline). In some embodiments of the pharmaceutical composition, the compounds of Formula (I), which may be compounds of Formula (II) and/or Formula (III), or salts thereof, can be incorporated into a liposome formulation.

In some embodiments, the pharmaceutical composition can further comprise an antigen in an amount effective to generate an immune response against the antigen. In some embodiments, the antigen is a vaccine.

The pharmaceutical composition can be administered in any suitable manner (parenterally or non-parenterally). In some embodiments, the pharmaceutical composition can be administered by an intradermal, subcutaneous, intramuscular, or intravenous injection.

In any embodiment of a pharmaceutical composition comprising a compound of Formula (II), the compound of Formula (II) is present in the composition in at least 80% enantiomeric excess, relative to the compound of Formula (III), at least 90% enantiomeric excess, at least 95% enantiomeric excess, at least 96% enantiomeric excess, at least 96% enantiomeric excess, at least 97% enantiomeric excess, at least 98% enantiomeric excess, at least 99% enantiomeric excess, at least 99.5% enantiomeric, or at least 99.8% enantiomeric excess.

In any embodiment of a pharmaceutical composition comprising a compound of Formula (III), the opposite enantiomer to the compound of Formula (II), is present in the composition in less than 10%, less than 5%, less than 2.5%, less than 2%, less than 1.5%, less than 1%, less than 0.5%, less than 0.25%, or less than 0.1%.

The exact amount of compound or salt used in a pharmaceutical composition of the disclosure will vary according to factors known to those of skill in the art, such as the physical and chemical nature of the compound or salt, the nature of the carrier, and the intended dosing regimen.

In some embodiments, the concentration of a compound of Formula (I), which may be a compound of Formula (II) and/or Formula (III), or salt thereof, in the pharmaceutical composition can be at least 0.0005 milligrams per milliliter (mg/mL), at least 0.001 mg/mL, or at least 0.05 mg/mL. In some embodiments, the concentration of a compound of Formula (I), which may be a compound of Formula (II) and/or Formula (III), or salt thereof, in the pharmaceutical composition can be up to 2.4 mg/mL, up to 0.06 mg/mL, up to 0.01 mg/mL, or up to 0.005 mg/mL.

In some embodiments, the compositions of the disclosure will contain sufficient active ingredient (i.e., compound of Formula (I) or salt thereof) or prodrug to provide a dose of at least 100 nanograms per kilogram (ng/kg), or at least 10 micrograms per kilogram (μg/kg), of the compound or salt to the subject. In some embodiments, the compositions of the disclosure will contain sufficient active ingredient (i.e., compound of Formula (I) or salt thereof) or prodrug to provide a dose of up to 50 milligrams per kilogram (mg/kg), or up to 5 mg/kg, of the compound or salt to the subject.

In some embodiments, the compositions of the disclosure will contain sufficient active ingredient (i.e., compound of Formula (I) or salt thereof) or prodrug to provide a dose of, for example, from 0.01 milligrams per square meter (mg/m²) to 5.0 mg/m², computed according to the Dubois method, in which the body surface area of a subject (m²) is computed using the subject's body weight: m²=(wt kg^{0.425}×height cm^{0.725})×0.007184, although in some embodiments the methods may be performed by administering a compound or salt or prodrug in a dose outside this range. In some of these embodiments, the method includes administering sufficient compound or salt or prodrug to provide a dose of from 0.1 mg/m² to 2.0 mg/m² to the subject, for example, a dose of from 0.4 mg/m² to 1.2 mg/m².

A variety of dosage forms may be used to administer the compounds or salts of the disclosure to a human or animal. Dosage forms that can be used include, for example, tablets, lozenges, capsules, parenteral formulations, creams, ointments, topical gels, aerosol formulations, liquid formulations (e.g., aqueous formulation), transdermal patches, and the like. These dosage forms can be prepared with conventional pharmaceutically acceptable carriers and additives using conventional methods, which generally include the step of bringing the active ingredient into association with the carrier. A preferred dosage form has one or more of compounds or salts of the disclosure dissolved in an aqueous formulation.

The compounds or salts described herein can be administered as the single therapeutic agent in the treatment regimen, or the compounds or salts described herein may be administered in combination with other active agents, including antivirals, antibiotics, proteins, peptides, oligonucleotides, antibodies, etc.

Compounds of Formula (I), particularly those of Formula (II), and salts thereof, induce the production of cytokines (e.g., IFN-alpha, IFN-gamma, TNF-alpha) in experiments performed according to the tests set forth below. These results indicate that Compounds of Formula (I), particularly those of Formula (II), and salts thereof, are useful for activating the immune response in a number of different ways, rendering them useful in the treatment of a variety of disorders. As such, the compounds of the disclosure, or salts thereof, are agonists of cytokine biosynthesis and production, particularly agonists of IFN-alpha, IFN-gamma, and TNF-alpha cytokine biosynthesis and production.

It is believed that one way in which the compounds or salts of the disclosure induce cytokine production is through the activation of Toll-like receptors (TLRs) in the immune system, particularly TLR-7 and/or TLR-8; however, other mechanisms may be involved. It is believed that in the immune system pathways (i.e., mechanisms) for cytokine induction, the compounds or salts of the disclosure, primarily act as agonists of TLR-7 and/or TLR-8, however, other pathways or activities may be involved.

Administration of compounds of Formula (I), particularly those of Formula (II), and salts thereof, can induce the production of interferon-alpha (IFN-alpha), interferon-gamma (IFN-gamma), and tumor necrosis factor-alpha (TNF-alpha) in cells. Cytokines whose biosynthesis can be induced by compounds of Formula (I), particularly those of Formula (II), and salts thereof include IFN-alpha, IFN-gamma, TNF-alpha, and a variety of other cytokines. Among other effects, these cytokines can inhibit virus production and tumor cell growth, making the compounds or salts useful in the treatment of viral diseases and neoplastic diseases.

Accordingly, the disclosure provides a method of inducing cytokine biosynthesis in a human or animal by administering an effective amount of a compound of Formula (I), particularly one of Formula (II), and salts thereof to the human or animal. The human or animal to which the compound or salt is administered for induction of cytokine production may have one or more diseases, disorders, or conditions described below, for example, a viral disease or a neoplastic disease, and administration of the compound or salt may provide therapeutic treatment. Alternatively, compounds of Formula (I), particularly those of Formula (II), and salts thereof may be administered to the human or animal prior to the human or animal acquiring the disease so that administration of the compound or salt may provide a prophylactic treatment.

In addition to the ability to induce the production of cytokines, compounds of Formula (I), particularly those of Formula (II), and salts thereof can affect other aspects of the innate immune response. For example, natural killer cell activity may be stimulated, an effect that may be due to cytokine induction. Compounds of Formula (I), particularly those of Formula (II), and salts thereof may also activate macrophages, which in turn stimulate secretion of nitric oxide and the production of additional cytokines. In addition, compounds of Formula (I), particularly those of Formula (II), and salts thereof may cause proliferation and differentiation of B-lymphocytes.

Conditions for which compounds of Formula (I), particularly those of Formula (II), and salts thereof may be used as treatment include, but are not limited to:

Viral diseases such as, for example, diseases resulting from infection by an adenovirus, a herpes virus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenza virus, avian influenza), a paramyxovirus (e.g., parainfluenza virus, mumps virus, measles virus, and respiratory syncytial virus (RSV), a coronavirus (e.g., SARS), a papovavirus (e.g., papillomaviruses, such as those that cause genital warts, common warts, or plantar warts), hepadnavirus (e.g., hepatitis B virus), a flavivirus (e.g., hepatitis C virus or Dengue virus), or a retrovirus (e.g., a lentivirus such as HIV), ebola virus;

Neoplastic diseases such as bladder cancer, cervical dysplasia, cervical cancer, actinic keratosis, basal cell carcinoma, cutaneous T-cell lymphoma, mycosis fungoides, Sezary Syndrome, HPV associated head and neck cancer (e.g., HPV positive oropharyngeal squamous cell carcinoma), Kaposi's sarcoma, melanoma, squamous cell carcinoma, renal cell carcinoma, acute myeloid leukemia, chronic myeloid leukemia, chronic lymphocytic leukemia, multiple myeloma, Hodgkin's lymphoma, non-Hodgkin's lymphoma, B-cell lymphoma, hairy cell leukemia, esophageal cancer, and other cancers;

T_{H}2-mediated atopic diseases such as atopic dermatitis or eczema, eosinophilia, asthma, allergy, allergic rhinitis, and Omenn's syndrome;

Diseases associated with wound repair, such as, for example, inhibition of keloid formation and other types of scarring (e.g., enhancing wound healing, including chronic wounds); and

Parasitic diseases including but not limited to malaria, leishmaniasis, cryptosporidiosis, toxoplasmosis, and trypanosome infection.

In addition, compounds of Formula (I), particularly those of Formula (II), and salts thereof, may be used as vaccine adjuvants for use in conjunction with any material that increases either humoral and/or cell mediated immune responses, such as, for example, tumor antigens (e.g., MAGE-3, NY-ESO-1); live viral, bacterial, or parasitic immunogens; inactivated viral, protozoal, fungal, or bacterial immunogens; toxoids; toxins; polysaccharides; proteins; glycoproteins; peptides; cellular vaccines; DNA vaccines; autologous vaccines; recombinant proteins; and the like.

Examples of vaccines that can benefit from use of compounds of Formula (I), particularly those of Formula (II), and salts thereof as vaccine adjuvants include BCG vaccine, cholera vaccine, plague vaccine, typhoid vaccine, hepatitis A vaccine, hepatitis B vaccine, hepatitis C vaccine, influenza A vaccine, influenza B vaccine, malaria vaccine, parainfluenza vaccine, polio vaccine, rabies vaccine, measles vaccine, mumps vaccine, rubella vaccine, yellow fever vaccine, tetanus vaccine, diphtheria vaccine, hemophilus influenza b vaccine, tuberculosis vaccine, meningococcal and pneumococcal vaccines, adenovirus vaccine, HIV vaccine, chicken pox vaccine, cytomegalovirus vaccine, dengue vaccine, feline leukemia vaccine, fowl plague vaccine, HSV-1 vaccine and HSV-2 vaccine, hog cholera vaccine, Japanese encephalitis vaccine, respiratory syncytial virus vaccine, rotavirus vaccine, papilloma virus vaccine, yellow fever vaccine, ebola virus vaccine.

Compounds of Formula (I), particularly those of Formula (II), and salts thereof may be particularly useful as vaccine adjuvants when used in conjunction with tumor antigens associated with colorectal cancer, head and neck cancer, breast cancer, lung cancer and melanoma.

Compounds of Formula (I), particularly those of Formula (II), and salts thereof may be particularly useful in individuals having compromised immune function. For example, compounds of Formula (I), particularly those of Formula (II), or salts thereof may be used for treating opportunistic infections and tumors that occur after suppression of cell mediated immunity in, for example, transplant patients, cancer patients, and HIV patients.

One or more of the above diseases or types of diseases, for example, a viral disease or neoplastic disease may be treated in a human or animal in need thereof (having the disease) by administering a therapeutically effective amount of a compound, salt, or composition to the human or animal.

A human or animal may also be vaccinated by administering an effective amount of a compound of Formula (I), particularly one of Formula (II), or a salt thereof, as a vaccine adjuvant. In one embodiment, a method of vaccinating a human or animal includes administering an effective amount of a compound of Formula (I), particularly one of Formula (II), or a salt thereof to the human or animal as a vaccine adjuvant. The vaccine adjuvant can be co-administered with the material that increases one or more humoral and cell mediated immune responses by including each in the same composition. Alternatively, the vaccine adjuvant and the material that increases either humoral and/or cell mediated immune responses can be in separate compositions.

Compounds of Formula (I), particularly those of Formula (II), or salts thereof may be used as prophylactic or therapeutic vaccine adjuvants in veterinary applications. Compounds of Formula (I), particularly those of Formula (II), or salts thereof may be administered to, for example, pigs, horses, cattle, sheep, dogs, cats, poultry (such as chickens or turkeys), etc.

Compounds of Formula (I), particularly those of Formula (II), or salts thereof may be particularly useful when an effective amount is administered to a human or animal to treat bladder cancer, cervical dysplasia, actinic keratosis, basal cell carcinoma, genital warts, herpes virus infection, or cutaneous T-cell lymphoma. For these conditions, administration of the Compounds of Formula (I), particularly those of Formula (II), or salts thereof is preferably topical (i.e., applied directly to the surface of a tumor, a lesion, a wart, or an infected tissue, etc.).

In one embodiment an effective amount of a compound of Formula (I), particularly one of Formula (II), or salt thereof in a composition such as an aqueous composition is administered into the bladder of a human or animal that has at least one tumor of the bladder by intravesical instillation (e.g., administration using a catheter).

An amount of a compound of Formula (I), particularly one of Formula (II), or salt thereof effective to induce cytokine biosynthesis will typically cause one or more cell types, such as monocytes, macrophages, dendritic cells, and B-cells to produce an amount of one or more cytokines, such as, for example, IFN-alpha, IFN-gamma, and TNF-alpha that is increased (induced) over a background level of such cytokines. The precise dose will vary according to factors known in the art but is typically to be a dose of 100 nanograms per kilogram (ng/kg) to 50 milligrams per kilogram (mg/kg), or 10 (micrograms per kilogram) μg/kg to 5 mg/kg. In other embodiments, the amount can be, for example, from 0.01 milligrams per square meter (mg/m²) to 5.0 mg/m² (computed according to the Dubois method as described above), although in other embodiments the induction of cytokine biosynthesis may be performed by administering a compound or salt in a dose outside this range. In some of these embodiments, the method includes administering sufficient compound or salt or composition to provide a dose from 0.1 mg/m² to 2.0 mg/m² to the subject, for example, a dose of from 0.4 mg/m² to 1.2 mg/m².

A method of treating a viral infection in a human or animal and a method of treating a neoplastic disease in a human or animal can include administering an effective amount of a compound of Formula (I), particularly one of Formula (II), or salt thereof to the human or animal.

An effective amount to treat or inhibit a viral infection can be an amount that will cause a reduction in one or more of the manifestations of viral infection, such as viral lesions, viral load, rate of virus production, and mortality as compared to untreated humans or animals. The precise amount that is effective for such treatment will vary according to factors known in the art but it is normally a dose of 100 ng/kg to 50 mg/kg, or 10 μg/kg to 5 mg/kg.

An effective amount to treat a neoplastic condition can be an amount that causes a reduction in tumor size or in the number of tumor foci. The precise amount will vary according to factors known in the art but is typically 100 ng/kg to 50 mg/kg, or 10 μg/kg to 5 mg/kg. In other embodiments, the amount is typically, for example, from 0.01 mg/m² to 5.0 mg/m² (computed according to the Dubois method as described above), although in some embodiments the induction of cytokine biosynthesis may be performed by administering a compound or salt in a dose outside this range. In some of these embodiments, the method includes administering sufficient compound or salt or composition to provide a dose from 0.1 mg/m² to 2.0 mg/m² to the subject, for example, a dose of from 0.4 mg/m² to 1.2 mg/m².

Compounds of Formula (I), particularly those of Formula (III), or salts thereof may be inactive toward cytokine production (e.g., the compounds of Example 2, Example 4, Example 6, Example 10, Example 12, Example 14, Example 16, Example 18, and Example 21) may be suitable in the treatment, e.g., of autoimmune conditions as a result of inhibiting cytokine biosynthesis. Thus, the present disclosure provides methods of inhibiting cytokine biosynthesis in a human or animal comprising administering an effective amount of one or more of such compounds to the human or animal. Effective amounts may be as described above and/or determined readily by one of skill in the art.

### EMBODIMENTS

Embodiment 1 is a compound of Formula (I), or salt thereof:

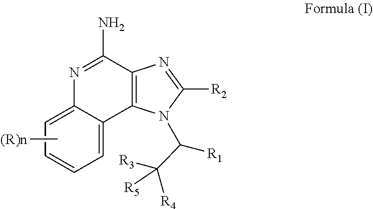

wherein:

n is an integer of 0 or 1;

R is selected from the group consisting of halogen, hydroxy, alkyl, alkoxy, and —C(O)—O-alkyl;

R₁ is a C₁₋₆alkyl;

R₂ is selected from the group consisting of hydrogen, methyl, ethyl, n-propyl, n-butyl, —CH₂OCH₃, —CH₂OCH₂CH₃, and —CH₂CH₂OCH₃;

R₅ is selected from the group consisting of —H, —CH₃, —F, and —OH; and

R₃ is a C₁₋₄alkyl, R₄ is a C₁₋₄alkyl, or R₃ and R₄ are combined to form a ring of 3-7 carbon atoms, optionally having one oxygen atom in the ring, provided that R₅ is not —OH (in certain embodiments, when R₅ is H, R₃ and R₄ may be combined to form a ring of 3-7 carbon atoms optionally having one oxygen atom in the ring).

Embodiment 2 is the compound or salt of embodiment 1, which is a compound of Formula (II), or salt thereof:

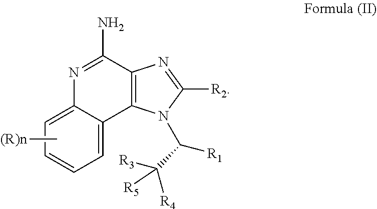

Embodiment 3 is the compound or salt of embodiment 1, which is a compound of Formula (III), or salt thereof:

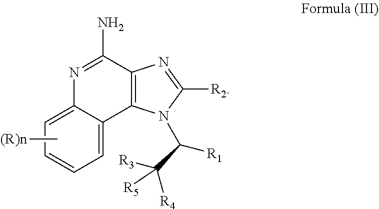

Embodiment 4 is the compound or salt of any of embodiments 1 through 3, wherein R is selected from the group consisting of halogen, hydroxy, —C₁₋₁₂alkyl, —C₁₋₁₂alkoxy, and —C(O)—O—C₁₋₁₀alkyl (in some embodiments, R is selected from the group consisting of halogen, hydroxy, —C₁₋₇alkyl, —C₁₋₇alkoxy, and —C(O)—O—C₁₋₅alkyl).

Embodiment 5 is the compound or salt of embodiment 4, wherein R is selected from the group consisting of hydroxy, F, and Cl.

Embodiment 6 is the compound or salt of embodiment 5, wherein R is selected from the group consisting of F and Cl.

Embodiment 7 is the compound or salt of any of embodiments 1 through 3, wherein n is 0.

Embodiment 8 is the compound or salt of any of embodiments 1 through 7, wherein R₁ is a C₁₋₄alkyl.

Embodiment 9 is the compound or salt of any of embodiments 1 through 7, wherein R₁ is a C₃₋₆alkyl (or C₃₋₄alkyl).

Embodiment 10 is the compound or salt of any of embodiments 1 through 9, wherein R₂ is hydrogen.

Embodiment 11 is the compound or salt of any of embodiments 1 through 9, wherein R₂ is selected from the group consisting of methyl, ethyl, n-propyl, and n-butyl.

Embodiment 12 is the compound or salt of any of embodiments 1 through 9, wherein R₂ is selected from the group consisting of —CH₂OCH₃, —CH₂OCH₂CH₃, and —CH₂CH₂OCH₃.

Embodiment 13 is the compound or salt of any of embodiments 1 through 9, wherein R₂ is selected from the group consisting of hydrogen, methyl, and ethyl.

Embodiment 14 is the compound or salt of any of embodiments 1 through 13, wherein R₃ is a C₁₋₄alkyl.

Embodiment 15 is the compound or salt of embodiment 14, wherein R₃ is methyl.

Embodiment 16 is the compound or salt of embodiment 14, wherein R₃ is ethyl.

Embodiment 17 is the compound or salt of any of embodiments 1 through 16, wherein R₄ is a C₁₋₄alkyl.

Embodiment 18 is the compound or salt of embodiment 17, wherein R₄ is methyl.

Embodiment 19 is the compound or salt of embodiment 17, wherein R₄ is ethyl.

Embodiment 20 is the compound or salt of embodiment 17, wherein R₃ and R₄ are each methyl.

Embodiment 21 is the compound or salt of embodiment 17, wherein R₃ and R₄ are each ethyl.

Embodiment 22 is the compound or salt of any of embodiments 1 through 21, wherein R₅ is selected from the group consisting of —H, —CH₃, and —F.

Embodiment 23 is the compound or salt of embodiments 22, wherein R₃ and R₄ are combined to form a ring of 3-7 carbon atoms optionally having one oxygen atom in the ring.

Embodiment 24 is the compound or salt of embodiment 23, wherein R₃ and R₄ are combined to form a ring of 3-7 carbon atoms.

Embodiment 25 is the compound or salt of embodiment 23, wherein R₃ and R₄ are combined to form a ring of 3-7 carbon atoms having one oxygen atom in the ring.

Embodiment 26 is the compound or salt of any of embodiments 1 through 25, wherein R₅ is —H.

Embodiment 27 is the compound or salt of any of embodiments 1 through 25, wherein R₅ is —CH₃.

Embodiment 28 is the compound or salt of any of embodiments 1 through 25, wherein R₅ is —F.

Embodiment 29 is the compound or salt of any of embodiments 1 through 21, wherein R₅ is —OH.

Embodiment 30 is the compound or salt of any of embodiments 1 through 3, wherein R₁ is a C₁₋₆alkyl (preferably, R₁ is selected from the group consisting of —CH₃, —CH₂CH₃, —CH₂CH₂CH₃, —CH₂CH₂CH₂CH₃, and —CH₂CH(CH₃)₂); R₂ is selected from the group consisting of hydrogen, methyl, and ethyl (preferably, R₂ is hydrogen); R₃ is a C₁₋₄alkyl; R₄ is a C₁₋₄alkyl; R₅ is selected from the group consisting of —H, —CH₃, —F, and —OH; and n is 0. In some embodiments of such compounds, R₅ is —H. In some embodiments of such compounds, R₅ is —F. In some embodiments of such compounds, R₅ is —OH. In some embodiments of such compounds, R₅ is —CH₃.

Embodiment 31 is the compound or salt of embodiment 30, wherein R₁ is selected from the group consisting of —CH₃, —CH₂CH₃, —CH₂CH₂CH₃, —CH₂CH₂CH₂CH₃, and —CH₂CH(CH₃)₂; R₂ is selected from the group consisting of hydrogen, methyl, and ethyl; R₃ is methyl or ethyl; R₄ is methyl or ethyl; R₅ is selected from the group consisting of

### —H, —CH₃, —F, and —OH; and n is 0.

Embodiment 32 is the compound or salt of embodiment 31, wherein R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is selected from the group consisting of

### —H, —CH₃, —F, and —OH; and n is 0.

Embodiment 33 is the compound or salt of embodiment 32, wherein R₁ is selected from the group consisting of —CH₃, —CH₂CH₃, —CH₂CH₂CH₃, —CH₂CH₂CH₂CH₃, and —CH₂CH(CH₃)₂, R₂ is hydrogen; R₃ is methyl or ethyl; R₄ is methyl or ethyl; R₅ is hydrogen; and n is 0.

Embodiment 34 is the compound or salt of embodiment 33, wherein R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl; R₄ is methyl; R₅ is hydrogen; and n is 0. Examples of such compounds include:
1-[(1R)-1,2-dimethylpropyl]imidazo[4,5-c]quinolin-4-amine (Example 1);
1-[(1S)-1,2-dimethylpropyl]imidazo[4,5-c]quinolin-4-amine (Example 2) and
1-[(1R)-1-isopropylpentyl]imidazo[4,5-c]quinolin-4-amine (Example 7).

Embodiment 35 is the compound or salt of embodiment 32, wherein R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is —CH₃; and n is 0. Examples of such compounds include:
1-[(1R)-1,2,2-trimethylpropyl]imidazo[4,5-c]quinolin-4-amine (Example 3);
1-[(1S)-1,2,2-trimethylpropyl]imidazo[4,5-c]quinolin-4-amine (Example 4) and
1-[(1R)-1-tert-butylpentyl]imidazo[4,5-c]quinolin-4-amine (Example 8).

Embodiment 36 is the compound or salt of embodiment 32, wherein R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is —F; and n is 0. Examples of such compounds include:
1-[(1R)-2-fluoro-1,2-dimethyl-propyl]imidazo[4,5-c]quinolin-4-amine (Example 20); and
1-[(1S)-2-fluoro-1,2-dimethyl-propyl]imidazo[4,5-c]quinolin-4-amine (Example 21).

Embodiment 37 is the compound or salt of embodiment 32, wherein R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is —OH; and n is 0. Examples of such compounds include:
(3R)-3-(4-aminoimidazo[4,5-c]quinolin-1-yl)-2-methyl-butan-2-ol (Example 9);
(3S)-3-(4-aminoimidazo[4,5-c]quinolin-1-yl)-2-methyl-butan-2-ol (Example 10);
(3R)-3-(4-aminoimidazo[4,5-c]quinolin-1-yl)-2-methyl-pentan-2-ol (Example 11);
(3S)-3-(4-aminoimidazo[4,5-c]quinolin-1-yl)-2-methyl-pentan-2-ol (Example 12);
(3R)-3-(4-aminoimidazo[4,5-c]quinolin-1-yl)-2-methyl-hexan-2-ol (Example 13);
(3S)-3-(4-aminoimidazo[4,5-c]quinolin-1-yl)-2-methyl-hexan-2-ol (Example 14);
(3R)-3-(4-aminoimidazo[4,5-c]quinolin-1-yl)-2-methyl-heptan-2-ol (Example 15);
(3S)-3-(4-aminoimidazo[4,5-c]quinolin-1-yl)-2-methyl-heptan-2-ol (Example 16);
(3R)-3-(4-aminoimidazo[4,5-c]quinolin-1-yl)-2,5-dimethyl-hexan-2-ol (Example 17);
(3S)-3-(4-aminoimidazo[4,5-c]quinolin-1-yl)-2,5-dimethyl-hexan-2-ol (Example 18) and
(2R)-2-(4-aminoimidazo[4,5-c]quinolin-1-yl)-3-ethyl-pentan-3-ol (Example 19).

Embodiment 38 is the compound or salt of any of embodiments 1 through 3, wherein R₁ C₁₋₆alkyl; R₂ is selected from the group consisting of hydrogen, methyl, and ethyl; R₃ and R₄ are combined to form a ring of 3-7 carbon atoms, optionally having one oxygen atom in the ring; R₅ is —H; and n is 0.

Embodiment 39 is the compound or salt of embodiment 38, wherein R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ and R₄ are combined to form a ring of 3-7 carbon atoms; R₅ is —H; and n is 0.

Examples of such compounds include: 1-[(1R)-1-cyclohexylethyl]imidazo[4,5-c]quinolin-4-amine (Example 5) and 1-[(1S)-1-cyclohexylethyl]imidazo[4,5-c]quinolin-4-amine (Example 6).

Embodiment 40 is the compound or salt of any of embodiments 1 through 39, which is a pharmaceutically acceptable salt.

Embodiment 41 is the compound or salt of embodiment 40, wherein the pharmaceutically acceptable salt is a hydrochloride salt.

Embodiment 42 is a pharmaceutical composition comprising an effective amount of a compound or salt of any of embodiments 1 through 41 in combination with a pharmaceutically acceptable carrier.

Embodiment 43 is the pharmaceutical composition of embodiment 42, wherein the compound of Formula (II) or salt thereof is present in at least 80%, at least 90%, at least 95%, at least 97%, or at least 98%, enantiomeric excess.

Embodiment 44 is the pharmaceutical composition of embodiment 43, wherein the compound of Formula (II) or salt thereof is present in at least 99% enantiomeric excess.

Embodiment 45 is the pharmaceutical composition of embodiment 44, wherein the compound of Formula (II) or salt thereof is present in at least 99.5% enantiomeric excess.

Embodiment 46 is the pharmaceutical composition of embodiment 45, wherein the compound of Formula (II) or salt thereof is present in at least 99.8% enantiomeric excess.

Embodiment 47 is the pharmaceutical composition of embodiment 42, wherein the compound of Formula (III) or salt thereof is present in at least 80%, at least 90%, at least 95%, at least 97%, or at least 98%, enantiomeric excess.

Embodiment 48 is the pharmaceutical composition of embodiment 47, wherein the compound of Formula (III) or salt thereof is present in at least 99% enantiomeric excess.

Embodiment 49 is the pharmaceutical composition of embodiment 48, wherein the compound of Formula (III) or salt thereof is present in at least 99.5% enantiomeric excess.

Embodiment 50 is the pharmaceutical composition of embodiment 49, wherein the compound of Formula (III) or salt thereof is present in at least 99.8% enantiomeric excess.

Embodiment 51 is the pharmaceutical composition of any of embodiments 42 through 46, further comprising an antigen.

Embodiment 52 is the pharmaceutical composition of any of embodiments 42 through 46 and 51 for use in treating an infectious disease in a human or animal.

Embodiment 53 is the pharmaceutical composition of embodiment 52 for use in treating a viral, bacterial, fungal, or parasitic infection in a human or animal.

Embodiment 54 is the pharmaceutical composition of any of embodiments 42 through 46 and 51 for use in treating a neoplastic disease in a human or animal.

Embodiment 55 is a method of inducing cytokine biosynthesis in a human or animal comprising administering an effective amount of a compound or salt of any of embodiments 1, 2, and 4 through 41, as dependent on embodiment 1 or 2, to the human or animal.

Embodiment 56 is the method of embodiment 55 comprising administering an effective amount of 1-[(1R)-1,2-dimethylpropyl]imidazo[4,5-c]quinolin-4-amine (Example 1).

Embodiment 57 is the method of embodiment 55 comprising administering an effective amount of 1-[(1R)-1,2,2-trimethylpropyl]imidazo[4,5-c]quinolin-4-amine (Example 3).

Embodiment 58 is the method of embodiment 55 comprising administering an effective amount of 1-[(1R)-1-cyclohexylethyl]imidazo[4,5-c]quinolin-4-amine (Example 5).

Embodiment 59 is the method of embodiment 55 comprising administering an effective amount of 1-[(1R)-1-isopropylpentyl]imidazo[4,5-c]quinolin-4-amine (Example 7).

Embodiment 60 is the method of embodiment 55 comprising administering an effective amount of 1-[(1R)-1-tert-butylpentyl]imidazo[4,5-c]quinolin-4-amine (Example 8).

Embodiment 61 is the method of embodiment 55 comprising administering an effective amount of (3R)-3-(4-aminoimidazo[4,5-c]quinolin-1-yl)-2-methyl-butan-2-ol (Example 9).

Embodiment 62 is the method of embodiment 55 comprising administering an effective amount of (3R)-3-(4-aminoimidazo[4,5-c]quinolin-1-yl)-2-methyl-pentan-2-ol (Example 11).

Embodiment 63 is the method of embodiment 55 comprising administering an effective amount of (3R)-3-(4-aminoimidazo[4,5-c]quinolin-1-yl)-2-methyl-hexan-2-ol (Example 13).

Embodiment 64 is the method of embodiment 55 comprising administering an effective amount of (3R)-3-(4-aminoimidazo[4,5-c]quinolin-1-yl)-2-methyl-heptan-2-ol (Example 15).

Embodiment 65 is the method of embodiment 55 comprising administering an effective amount of (3R)-3-(4-aminoimidazo[4,5-c]quinolin-1-yl)-2,5-dimethyl-hexan-2-ol (Example 17).

Embodiment 66 is the method of embodiment 55 comprising administering an effective amount of (2R)-2-(4-aminoimidazo[4,5-c]quinolin-1-yl)-3-ethyl-pentan-3-ol (Example 19).

Embodiment 67 is the method of embodiment 55 comprising administering an effective amount of 1-[(1R)-2-fluoro-1,2-dimethyl-propyl]imidazo[4,5-c]quinolin-4-amine (Example 20).

Embodiment 68 is the method of any of embodiments 55 through 67, wherein the cytokine is IFN-alpha.

Embodiment 69 is the method of any of embodiments 55 through 67, wherein the cytokine is IFN-gamma.

Embodiment 70 is the method of any of embodiments 55 through 67, wherein the cytokine is TNF-alpha.

Embodiment 71 is a method of inhibiting cytokine biosynthesis in a human or animal comprising administering an effective amount of a compound or salt of any of embodiments 1, 3, and 4 through 41 as dependent on embodiment 1 or 3, to the human or animal.

Embodiment 72 is the method of embodiment 71 comprising administering an effective amount of 1-[(1S)-1,2-dimethylpropyl]imidazo[4,5-c]quinolin-4-amine (Example 2).

Embodiment 73 is the method of embodiment 71 comprising administering an effective amount of 1-[(1S)-1,2,2-trimethylpropyl]imidazo[4,5-c]quinolin-4-amine (Example 4).

Embodiment 74 is the method of embodiment 71 comprising administering an effective amount of 1-[(1S)-1-cyclohexylethyl]imidazo[4,5-c]quinolin-4-amine (Example 6).

Embodiment 75 is the method of embodiment 71 comprising administering an effective amount of (3S)-3-(4-aminoimidazo[4,5-c]quinolin-1-yl)-2-methyl-butan-2-ol (Example 10).

Embodiment 76 is the method of embodiment 71 comprising administering an effective amount of (3S)-3-(4-aminoimidazo[4,5-c]quinolin-1-yl)-2-methyl-pentan-2-ol (Example 12).

Embodiment 77 is the method of embodiment 71 comprising administering an effective amount of (3S)-3-(4-aminoimidazo[4,5-c]quinolin-1-yl)-2-methyl-hexan-2-ol (Example 14).

Embodiment 78 is the method of embodiment 71 comprising administering an effective amount of (3S)-3-(4-aminoimidazo[4,5-c]quinolin-1-yl)-2-methyl-heptan-2-ol (Example 16).

Embodiment 79 is the method of embodiment 71 comprising administering an effective amount of (3S)-3-(4-aminoimidazo[4,5-c]quinolin-1-yl)-2,5-dimethyl-hexan-2-ol (Example 18).

Embodiment 80 is the method of embodiment 71 comprising administering an effective amount of 1-[(1S)-2-fluoro-1,2-dimethyl-propyl]imidazo[4,5-c]quinolin-4-amine (Example 21).

Embodiment 81 is a compound or salt of any of embodiments 1, 2, and 4 through 41, as dependent on embodiment 1 or 2, for use as a vaccine adjuvant in treating an infectious disease in a human or animal.

Embodiment 82 is a compound or salt of any of embodiments 1, 2, and 4 through 41, as dependent on embodiment 1 or 2, for use as a vaccine adjuvant in treating a viral, bacterial, fungal, or parasitic infection in a human or animal.

Embodiment 83 is a compound or salt of embodiment 81 or 82, wherein the treatment is a therapeutic or prophylactic treatment.

Embodiment 84 is a method of treating a neoplastic disease in a human or animal by administering an effective amount of a compound or salt of any of embodiments 1, 2, and 4 through 41, as dependent on embodiment 1 or 2, to the human or animal.

Embodiment 85 is the method of embodiment 84 wherein the neoplastic disease is selected from bladder cancer, cervical dysplasia, cervical cancer, actinic keratosis, basal cell carcinoma, cutaneous T-cell lymphoma, mycosis fungoides, Sezary Syndrome, HPV associated head and neck cancer (e.g., HPV positive oropharyngeal squamous cell carcinoma), Kaposi's sarcoma, melanoma, squamous cell carcinoma, renal cell carcinoma, acute myeloid leukemia, chronic myeloid leukemia, chronic lymphocytic leukemia, multiple myeloma, Hodgkin's lymphoma, non-Hodgkin's lymphoma, B-cell lymphoma, hairy cell leukemia, esophageal cancer, and combinations thereof.

### EXAMPLES

Objects and advantages of the disclosure are further illustrated by the examples provided herein. The particular materials and amounts thereof recited in these examples, as well as other conditions and details, are merely illustrative and are not intended to be limiting. The person of ordinary skill in the art, after carefully reviewing the entirety of this disclosure, will be able to use materials and conditions in addition to those specifically described in the examples.

Automated flash chromatography (AFC) was carried out using an ISOLARA HPFC system (an automated high-performance flash purification product available from Biotage Incorporated, Charlottesville, Va.). The eluent used for each purification is given in the example. In some chromatographic separations, the solvent mixture 80/18/2 v/v/v chloroform/methanol/concentrated ammonium hydroxide (CMA) was used as the polar component of the eluent. In these separations, CMA was mixed with chloroform in the indicated ratio.

Proton nuclear magnetic resonance (¹H NMR) analysis was conducted using a BRUKER A500 NMR spectrometer (Bruker Corporation, Bilerica, Mass.).

Ten percent (10%) palladium on carbon, 3-chloroperbenzoic acid (57-86%, MCPBA), allyl magnesium bromide in diethyl ether (1.0 M), sodium borohydride, thionyl chloride, (2,2,6,6-tetramethylpiperidin-1-yl)oxyl (TEMPO), L-alanine methyl ester hydrochloride and L-alanine methyl ester hydrochloride were obtained from the Sigma-Aldrich Company, St. Louis, Mo.

Triethyl orthoformate, 3% platinum on carbon, n-propyl acetate, para-toluenesulfonyl chloride, methyl magnesium bromide in diethyl ether (3.0 M), ethyl magnesium bromide in diethyl ether (3.0 M), (2R)-3-methylbutan-2-amine, (2S)-3-methylbutan-2-amine, (2R)-3,3-dimethylbutan-2-amine, (2S)-3,3-dimethylbutan-2-amine, (1R)-cyclohexyl ethylamine, (1S)-cyclohexyl ethylamine, diethylamino sulfur trifluoride (DAST) and pyridine hydrochloride were obtained from the Alfa Aesar Company, Haverhill, Mass.

L-tert-leucine, (R)-2-aminobutyric acid, (S)-2-aminobutyric acid, (R)-2-aminopentanoic acid, (S)-2-aminopentanoic acid, (R)-2-aminohexanoic acid, (S)-2-aminohexanoic acid, D-leucine, L-leucine, di-tert-butyl dicarbonate, propyltriphenylphosphonium bromide, 11% solution of potassium bis(trimethylsilyl)amide in toluene and 3-chloroperbenzoic acid (80%, MCPBA) were obtained from Oakwood Products Incorporated, Estill, S.C.

L-valinol, was obtained from TCI America, Portland, Oreg.

Iodine was obtained from Mallinckrodt, Inc., St. Louis Mo.

Sodium bromide, potassium iodide and sodium thiosulfate 0.1 N volumetric solution were obtained from J.T. Baker Chemical Co. Phillipsburg, N.J.

Triethylamine was obtained from EMD Millipore Corporation, Darmstadt Germany.

p-Toluene sulfonic acid monohydrate was obtained from Fisher Scientific Company, Fair Lawn, N.J.

CLOROX bleach was the source of sodium hypochlorite solution and was obtained from The Clorox Company, Oakland, Calif. The sodium hypochlorite concentration was determined by titration using iodine and sodium thiosulfate 0.1 N volumetric solution.

### Example 1

### 1-[(1R)-1,2-dimethylpropyl]imidazo[4,5-c]quinolin-4-amine

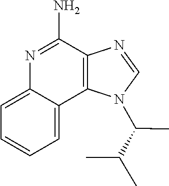 [SMILES: CC(C)[C@@H](C)n1cnc2c(N)nc3ccccc3c21]

### Part A

A solution of (2R)-3-methylbutan-2-amine (2.09 grams (g), 24.0 millimole (mmol)) in 100 milliliters (mL) of methylene chloride was combined with 4-chloro-3-nitroquinoline (5.00 g, 24.0 mmol) and triethylamine (5.09 g, 50.4 mmol) and the reaction mixture was stirred under an atmosphere of nitrogen for 2 days. Saturated aqueous K₂CO₃ solution (50 mL) was added followed by 150 mL of deionized water. The layers were separated and the aqueous portion was further extracted with methylene chloride. The combined organic portions were dried over Na₂SO₄, filtered and concentrated to give a yellow solid. The yellow solid was stirred with K₂CO₃ solution (50 mL) and 400 mL of deionized water, filtered and dried to give 6.20 g of N-[(1R)-1,2-dimethylpropyl]-3-nitro-quinolin-4-amine as a yellow solid.

### Part B

A solution of N-[(1R)-1,2-dimethylpropyl]-3-nitro-quinolin-4-amine (6.20 g, 23.9 mmol) dissolved in 250 mL of toluene was placed in a pressure bottle followed by addition of 500 milligrams (mg) of 5% platinum on carbon and MgSO₄ (1 g). The bottle was then shaken under an atmosphere of hydrogen (50 pounds per square inch (PSI)) for 12 hours. The reaction mixture was filtered through a pad of CELITE and the filtrate was concentrated under reduced pressure to give 5.37 g of N4-[(1R)-1,2-dimethylpropyl]quinoline-3,4-diamine.

### Part C

A solution of N4-[(1R)-1,2-dimethylpropyl]quinoline-3,4-diamine (5.37 g, 23.4 mmol) dissolved in 100 mL of toluene was combined with diethoxymethyl acetate (3.69 g, 22.7 mmol) in a round bottom flask equipped with a Dean-Stark trap and the mixture was heated to reflux. The first 10 mL of distillate in the Dean-Stark trap was removed and heating was continued overnight. The cooled reaction mixture was diluted with 50 mL of ethyl acetate and washed successively with saturated NaHCO₃ solution, water and brine. The reaction mixture was the concentrated under reduced pressure to give 5.43 g of 1-[(1R)-1,2-dimethylpropyl]imidazo[4,5-c]quinoline as a light brown oil.

### Part D

A solution of 1-[(1R)-1,2-dimethylpropyl]imidazo[4,5-c]quinoline (5.43 g, 22.7 mmol) dissolved in 50 mL of chloroform was combined with 6.27 g of MCPBA (60%) and stirred overnight. The reaction mixture was treated with 1% Na₂CO₃ solution and the layers were separated. The aqueous portion was further extracted with two 50 mL portions of chloroform and the combined organic portions were dried over Na₂SO₄, filtered and concentrated to give a red solid. The red solid was suspended in 100 mL of methylene chloride and the mixture was stirred rapidly. Concentrated NH₄OH solution (35 mL) and p-toluenesulfonyl chloride (4.33 g, 22.7 mmol) were then added. After stirring for 2 days, the reaction mixture was transferred to a separatory funnel and the layers were separated. The aqueous portion was further extracted with two 50 mL portions of methylene chloride and the combined organic portions were washed with 100 mL of 1% Na₂CO₃ solution. The organic portion was dried over MgSO₄, filtered and concentrated to give a white solid. The white solid was slurried in 100 mL of hot acetonitrile, filtered, washed with cold acetonitrile and dried under vacuum to give 3.45 g of 1-[(1R)-1,2-dimethylpropyl]imidazo[4,5-c]quinolin-4-amine as a white powder.

¹H NMR (500 MHz, METHANOL-d₄) δ 8.36 (s, 1H), 8.17 (dd, J=1.0, 8.3 Hz, 1H), 7.72 (dd, J=1.0, 8.4 Hz, 1H), 7.51 (ddd, J=1.3, 7.1, 8.3 Hz, 1H), 7.35 (ddd, J=1.3, 7.1, 8.3 Hz, 1H), 5.06 (br s, 1H), 2.36 (qd, J=6.6, 13.3 Hz, 1H), 1.71 (d, J=6.9 Hz, 3H), 0.93-1.04 (m, 6H).

### Example 2

### 1-[(1S)-1,2-dimethylpropyl]imidazo[4,5-c]quinolin-4-amine

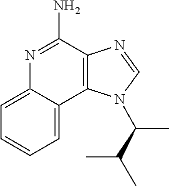 [SMILES: CC(C)[C@H](C)n1cnc2c(N)nc3ccccc3c21]

This compound was prepared from (2S)-3-methylbutan-2-amine following the procedures described in Parts A-D for Example 1.

¹H NMR (500 MHz, METHANOL-d₄) δ 8.37 (s, 1H), 8.18 (dd, J=0.9, 8.3 Hz, 1H), 7.72 (dd, J=0.9, 8.4 Hz, 1H), 7.52 (ddd, J=1.3, 7.1, 8.3 Hz, 1H), 7.36 (ddd, J=1.3, 7.1, 8.3 Hz, 1H), 5.08 (br s, 1H), 2.32-2.43 (m, 1H), 1.72 (d, J=6.9 Hz, 3H), 0.95-1.04 (m, 6H).

### Example 3

### 1-[(1R)-1,2,2-trimethylpropyl]imidazo[4,5-c]quinolin-4-amine

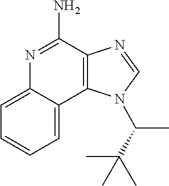 [SMILES: C[C@@H](n1cnc2c(N)nc3ccccc3c21)C(C)(C)C]

### Part A

A solution of (2R)-3,3-dimethylbutan-2-amine (700 mg, 6.92 mmol) in 25 mL of methylene chloride was combined with 4-chloro-3-nitroquinoline (1.31 g, 6.34 mmol) and triethylamine (2.65 mL, 19.0 mmol) and the reaction mixture was stirred under an atmosphere of nitrogen overnight. The reaction mixture was concentrated to give a yellow solid. The solid was dissolved in 50 mL of warm ethyl acetate and washed successively with water (2×) and brine. The organic portion was dried over Na₂SO₄, filtered and concentrated to give 1.68 g of 3-nitro-N-[(1R)-1,2,2-trimethylpropyl]quinolin-4-amine as a yellow solid.

### Part B

A solution of 3-nitro-N-[(1R)-1,2,2-trimethylpropyl]quinolin-4-amine (1.68 g, 6.15 mmol) dissolved in 30 mL acetonitrile was placed in a pressure bottle followed by addition of 100 mg of 3% platinum on carbon. The bottle was then shaken under an atmosphere of hydrogen (40 PSI) for 2 hours. The reaction mixture was filtered through a pad of CELITE and the filtrate was concentrated under reduced pressure to give 1.51 g of N4-[(1R)-1,2,2-trimethylpropyl]quinoline-3,4-diamine as an orange solid.

### Part C

A solution of N4-[(1R)-1,2,2-trimethylpropyl]quinoline-3,4-diamine (1.51 g, 6.21 mmol) dissolved in 30 mL of n-propyl acetate was combined with triethyl orthoformate (1.55 mL, 9.32 mmol) and 100 mg of pyridine hydrochloride and the mixture was heated to 90° C. overnight. The cooled reaction mixture was diluted with 50 mL of ethyl acetate and washed successively with saturated NaHCO₃ solution, water and brine. The organic portion was dried over Na₂SO₄, filtered and concentrated to give a light brown foam. Purification by column chromatography (SiO₂, 1% methanol/chloroform-10% methanol/chloroform) gave 1.03 g of (3R)-1-[(1R)-1,2,2-trimethylpropyl]imidazo[4,5-c]quinoline as a light brown solid.

### Part D

A solution of (3R)-1-[(1R)-1,2,2-trimethylpropyl]imidazo[4,5-c]quinoline (1.03 g, 4.04 mmol) dissolved in 25 mL of methylene chloride was combined with 1.30 g of MCPBA (57-86%) and stirred for 40 minutes. The reaction mixture was treated with 2% Na₂CO₃ solution and the layers were separated. The aqueous portion was further extracted with two 25 mL portions of methylene chloride and the combined organic portions were dried over Na₂SO₄, filtered and concentrated to give 1.09 g of an orange solid. The orange solid was suspended in 40 mL of methylene chloride and the mixture was stirred rapidly. Concentrated NH₄OH solution (10 mL) and p-toluenesulfonyl chloride (846 mg, 4.44 mmol) were then added. After stirring for 45 minutes, the reaction mixture was diluted with 25 mL of methylene chloride and washed successively with water (3×) and brine. The organic portion was dried over Na₂SO₄, filtered and concentrated under reduced pressure. Purification by column chromatography (SiO₂, 10% CMA/chloroform-50% CMA/chloroform) gave a light brown syrup which was crystallized from 2-propanol to give 390 mg of 1-[(1R)-1,2,2-trimethylpropyl]imidazo[4,5-c]quinolin-4-amine as amber crystals.

¹H NMR (500 MHz, METHANOL-d₄) δ 8.38 (s, 1H), 8.38 (dd, J=1.0, 8.4 Hz, 1H), 7.73 (dd, J=1.0, 8.4 Hz, 1H), 7.51 (ddd, J=1.3, 7.1, 8.3 Hz, 1H), 7.36 (ddd, J=1.3, 7.0, 8.3 Hz, 1H), 5.31 (q, J=7.1 Hz, 1H), 1.74 (d, J=7.1 Hz, 3H), 1.02 (s, 9H).

### Example 4

### 1-[(1S)-1,2,2-trimethylpropyl]imidazo[4,5-c]quinolin-4-amine

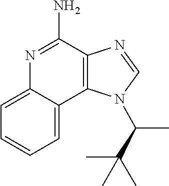 [SMILES: C[C@H](n1cnc2c(N)nc3ccccc3c21)C(C)(C)C]

This compound was prepared from (2S)-3,3-dimethylbutan-2-amine following the procedures described in Parts A-D for Example 3.

¹H NMR (500 MHz, METHANOL-d₄) δ 8.38 (s, 1H), 8.38 (dd, J=1.0, 8.4 Hz, 1H), 7.73 (dd, J=1.0, 8.3 Hz, 1H), 7.52 (dt, J=1.2, 7.7 Hz, 1H), 7.37 (dt, J=1.2, 7.6 Hz, 1H), 5.33 (q, J=7.1 Hz, 1H), 1.76 (d, J=7.1 Hz, 3H), 1.04 (s, 9H).

### Example 5

### 1-[(1R)-1-cyclohexylethyl]imidazo[4,5-c]quinolin-4-amine

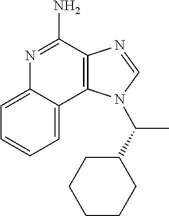 [SMILES: C[C@H](C1CCCCC1)n1cnc2c(N)nc3ccccc3c21]

### Part A

A solution of (1R)-cyclohexyl ethylamine (1.79 g, 14.1 mmol) dissolved in 30 mL of methylene chloride was combined with 4-chloro-3-nitroquinoline (2.66 g, 12.8 mmol) and triethylamine (3.56 mL, 25.6 mmol) and the reaction mixture was stirred under an atmosphere of nitrogen overnight. The reaction mixture was concentrated to give a yellow solid. The solid was dissolved in 75 mL of ethyl acetate and washed successively with water (2×) and brine. The organic portion was dried over Na₂SO₄, filtered and concentrated. Purification by column chromatography (SiO₂, 3% methanol/chloroform) gave 3.55 g of N-[(1R)-1-cyclohexylethyl]-3-nitro-quinolin-4-amine as a yellow syrup.

### Part B

A solution of N-[(1R)-1-cyclohexylethyl]-3-nitro-quinolin-4-amine (3.55 g, 11.9 mmol) dissolved in 30 mL acetonitrile was placed in a pressure bottle followed by addition of 100 mg of 3% platinum on carbon. The bottle was then shaken under an atmosphere of hydrogen (40 PSI) for 2 hours. The reaction mixture was filtered through a pad of CELITE and the filtrate was concentrated under reduced pressure to give 3.19 g of N4-[(1R)-1-cyclohexylethyl]quinoline-3,4-diamine as a mauve solid.

### Part C

A solution of N4-[(1R)-1-cyclohexylethyl]quinoline-3,4-diamine (3.19 g, 11.9 mmol) dissolved in 75 mL of n-propyl acetate was combined with triethyl orthoformate (2.96 mL, 17.8 mmol) and 200 mg of pyridine hydrochloride and the mixture was heated to 90° C. overnight. The cooled reaction mixture was diluted with 75 mL of ethyl acetate and washed successively with saturated NaHCO₃ solution, water and brine. The organic portion was dried over Na₂SO₄, filtered and concentrated to give a brown syrup. Purification by column chromatography (SiO₂, 1% methanol/chloroform-10% methanol/chloroform) gave 2.59 g of 1-[(1R)-1-cyclohexylethyl]imidazo[4,5-c]quinoline as a mauve syrup.

### Part D

A solution of 1-[(1R)-1-cyclohexylethyl]imidazo[4,5-c]quinoline (2.59 g, 9.28 mmol) dissolved in 25 mL of methylene chloride was combined with 2.80 g of MCPBA (57-86%) and stirred for 60 minutes. The reaction mixture was combined with 2% Na₂CO₃ solution and the layers were separated. The aqueous portion was further extracted with two 25 mL portions of methylene chloride and the combined organic portions were washed with brine, dried over Na₂SO₄, filtered and concentrated to give 2.60 g of a brown syrup. The brown syrup was suspended in 40 mL of methylene chloride and the mixture was stirred rapidly. Concentrated NH₄OH solution (10 mL) and p-toluenesulfonyl chloride (1.95 g, 10.2 mmol) were then added. After stirring for 45 minutes, the reaction mixture was diluted with 25 mL of methylene chloride and washed successively with water (3×) and brine. The organic portion was dried over Na₂SO₄, filtered and concentrated under reduced pressure. Purification by column chromatography (SiO₂, 10% CMA/chloroform-50% CMA/chloroform) gave a light brown syrup. The syrup was dissolved in 20 mL of ethanol and 1 mL of concentrated hydrochloric acid and the mixture was concentrated to dryness. Crystallization from 2-propanol/water gave 734 mg of 1-[(1R)-1-cyclohexylethyl]imidazo[4,5-c]quinolin-4-amine hydrochloride as colorless needles.

¹H NMR (500 MHz, METHANOL-d₄) δ 8.57 (s, 1H), 8.39 (d, J=7.95 Hz, 1H), 7.81-7.87 (m, 1H), 7.77 (dt, J=1.1, 7.8 Hz, 1H), 7.66 (dt, J=1.2, 7.8 Hz, 1H), 5.04-5.28 (m, 1H), 1.96-2.08 (m, 1H), 1.65-1.89 (m, 4H), 1.78 (d, J=6.9 Hz, 3H), 1.59 (m, 1H), 1.07-1.34 (m, 5H).

### Example 6

### 1-[(1S)-1-cyclohexylethyl]imidazo[4,5-c]quinolin-4-amine

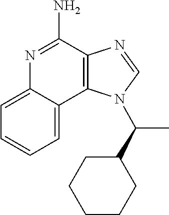 [SMILES: C[C@@H](C1CCCCC1)n1cnc2c(N)nc3ccccc3c21]

This compound was prepared from (1S)-cyclohexyl ethylamine following the procedures described in Parts A-D for Example 5 with the exception that the final compound was isolated as the free base by crystallization from acetonitrile.

¹H NMR (500 MHz, DEUTERIUM OXIDE) δ 8.43 (br s, 1H), 8.24 (br d, J=8.31 Hz, 1H), 7.67-7.74 (m, 1H), 7.59-7.65 (m, 1H), 7.48-7.55 (m, 1H), 4.99 (m, 1H), 1.86 (br s, 1H), 1.50-1.74 (m, 4H), 1.64 (d, J=6.9 Hz, 3H), 1.43 (br s, 1H), 0.86-1.20 (m, 5H).

### Example 7

### 1-[(1R)-1-isopropylpentyl]imidazo[4,5-c]quinolin-4-amine

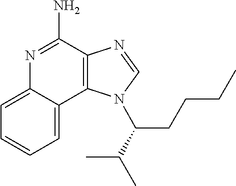 [SMILES: CCCC[C@H](C(C)C)n1cnc2c(N)nc3ccccc3c21]

### Part A

A solution of L-valinol (5.20 g, 50.5 mmol) dissolved in 60 mL of methylene chloride was combined with triethylamine (7.73 mL, 55.5 mmol) and di-tert-butyl decarbonate (11.0 g, 50.5 mmol). After stirring for 2 days at ambient temperature, a solution of 10% aqueous citric acid (100 mL) was added and the layers were separated. The organic portion was washed successively with an additional portion of 10% citric acid solution, water and brine. The organic portion was dried over Na₂SO₄, filtered and concentrated to give 9.83 g of tert-butyl N-[(1S)-1-(hydroxymethyl)-2-methyl-propyl]carbamate as a colorless syrup.

### Part B

A solution of tert-butyl N-[(1S)-1-(hydroxymethyl)-2-methyl-propyl]carbamate (2.03 g, 10.0 mmol) dissolved in 60 mL of a 1:1 mixture of ethyl acetate/toluene was placed in a round bottom flask. A solution of sodium bromide (1.08 g, 10.5 mmol) dissolved in 5 mL of deionized water was then added to the flask and the mixture was stirred in a −2° C. bath. TEMPO (22 mg) was then added to the stirred mixture followed by the dropwise addition of a solution containing aqueous sodium hypochlorite (4.4% by weight, 18.6 g, 11.0 mmol) and NaHCO₃ (2.56 g, 30 mmol) dissolved in 20 mL of deionized water. After addition was complete, the mixture was stirred for an additional 20 minutes. The mixture was then diluted with ethyl acetate (20 mL) and transferred to a separatory funnel and the layers were separated. The aqueous layer was extracted with an additional 20 mL portion of ethyl acetate. The combined organic portions were successively washed with 30 mL of 10% aqueous citric acid containing 360 mg of potassium iodide, 10% aqueous Na₂S₂O₃, water and finally brine. The organic portion was dried over Na₂SO₄, filtered and concentrated to give 1.59 g of tert-butyl N-[(1S)-1-formyl-2-methyl-propyl]carbamate as a light golden oil.

### Part C

A dry 250 mL round bottom flask was charged with propyltriphenylphosphonium bromide (3.05 g, 7.93 mmol) and 30 mL of anhydrous toluene. The reaction mixture was cooled in a 0° C. bath and stirred under a nitrogen atmosphere. An 11% solution of potassium bis(trimethylsilyl)amide in toluene (14.4 g, 7.93 mmol) was then added to the flask. After stirring for 15 minutes the reaction mixture was transferred to a −78° C. bath and a solution of tert-butyl N-[(1S)-1-formyl-2-methyl-propyl]carbamate (1.59 g, 7.93 mmol) dissolved in 15 mL of anhydrous toluene was added. The stirred mixture was allowed to warm to ambient temperature overnight. The reaction with was quenched by addition of saturated NH₄Cl solution followed by addition of 30 mL of diethyl ether. The layers were separated and the aqueous portion was extracted with an additional 20 mL of diethyl ether. The combined organic layers were washed with brine, dried over MgSO₄, filtered and concentrated under reduced pressure. The resulting material was combined with 25% ethyl acetate/hexanes to precipitate triphenylphosphine oxide which was removed by filtering through a plug of silica gel eluting with 25% ethyl acetate/hexanes. The eluate was concentrated to give a colorless semi-solid. Purification by column chromatography (SiO₂, 10% ethyl acetate/hexanes) gave 1.47 g of tert-butyl N—[(Z,1S)-1-isopropylpent-2-enyl]carbamate as a white solid.

### Part D

A solution of tert-butyl N—[(Z,1S)-1-isopropylpent-2-enyl]carbamate (1.47 g) dissolved in 25 mL methanol was placed in a pressure bottle followed by addition of 200 mg of 10% palladium on carbon. The bottle was then shaken under an atmosphere of hydrogen (40 PSI) overnight. The reaction mixture was filtered through a pad of CELITE and the filtrate was concentrated under reduced pressure to give 1.46 g of tert-butyl N-[(1R)-1-isopropylpentyl]carbamate as a colorless oil.

### Part E

A solution of tert-butyl N-[(1R)-1-isopropylpentyl]carbamate (1.46 g, 6.38 mmol) dissolved in 20 mL of ethanol was combined with 2 mL of concentrated hydrochloric acid. The stirred reaction mixture was heated to reflux for 2 hours and then concentrated under reduced pressure to give an oil. Crystallization from acetonitrile gave 762 mg of (3R)-2-methylheptan-3-amine hydrochloride as white needles.

### Part F

A solution of (3R)-2-methylheptan-3-amine hydrochloride (762 mg, 4.60 mmol) dissolved in 25 mL of methylene chloride was combined with 4-chloro-3-nitroquinoline (959 mg, 4.60 mmol) and triethylamine (1.92 mL, 13.8 mmol) and the reaction mixture was stirred under an atmosphere of nitrogen overnight. The reaction mixture was concentrated to give a yellow solid. The solid was dissolved in 75 mL of ethyl acetate and washed successively with water and brine. The organic portion was dried over Na₂SO₄, filtered and concentrated to give a yellow syrup. Purification by column chromatography (SiO₂, 2% methanol/chloroform) gave 0.75 g of N-[(1R)-1-isopropylpentyl]-3-nitro-quinolin-4-amine as a yellow syrup.

### Part G

A solution of N-[(1R)-1-isopropylpentyl]-3-nitro-quinolin-4-amine (0.75 g, 2.49 mmol) dissolved in 15 mL acetonitrile was placed in a pressure bottle followed by addition of 100 mg of 3% platinum on carbon. The bottle was then shaken under an atmosphere of hydrogen (40 PSI) overnight. An additional 100 mg of 3% platinum on carbon was added to the reaction and shaking under an atmosphere of hydrogen (40 PSI) was continued for 4 hours. The reaction mixture was filtered through a pad of CELITE and the filtrate was concentrated under reduced pressure to give 664 mg of N4-[(1R)-1-isopropylpentyl]quinoline-3,4-diamine as a yellow syrup.

### Part H

A solution of N4-[(1R)-1-isopropylpentyl]quinoline-3,4-diamine (664 mg, 2.45 mmol) dissolved in 25 mL of n-propyl acetate was combined with triethyl orthoformate (1.32 mL, 7.35 mmol) and 50 mg of pyridine hydrochloride and the mixture was heated to 100° C. overnight. The warm reaction mixture was washed successively with saturated NaHCO₃ solution, water and brine. The organic portion was dried over Na₂SO₄, filtered and concentrated to give a brown syrup. Purification by column chromatography (SiO₂, 3% methanol/chloroform₃) gave 671 mg 1-[(1R)-1-isopropylpentyl]imidazo[4,5-c]quinoline as a yellow syrup.

### Part I

A solution of 1-[(1R)-1-isopropylpentyl]imidazo[4,5-c]quinoline (671 mg, 2.39 mmol) dissolved in 20 mL of methylene chloride was combined with 539 mg of MCPBA (80%) and stirred for 50 minutes. The reaction mixture was combined with 10% Na₂CO₃ solution and 10 mL of methylene chloride and the layers were separated. The aqueous portion was further extracted with two additional 10 mL portions of methylene chloride. The combined organic layers were washed with brine, dried over Na₂SO₄, filtered and concentrated to give an amber foam. A stirred solution of the amber foam dissolved in 20 mL of methylene chloride was combined with 5 mL of concentrated NH₄OH solution and p-toluenesulfonyl chloride (501 mg, 2.63 mmol). After stirring for 45 minutes, the reaction mixture was diluted with 30 mL of methylene chloride and washed successively with water (3×) and brine. The organic portion was dried over Na₂SO₄, filtered and concentrated under reduced pressure. Purification by column chromatography (SiO₂, 3.3% methanol/chloroform saturated with NH₄OH) gave a light brown foam. The light brown foam was dissolved in 5 mL of ethanol and 1 mL of concentrated hydrochloric acid. The mixture was evaporated to dryness. Crystallization from acetonitrile gave 217 mg of 1-[(1R)-1-isopropylpentyl]imidazo[4,5-c]quinolin-4-amine hydrochloride as a cream colored powder.

¹H NMR (500 MHz, METHANOL-d₄) δ 8.59 (s, 1H), 8.52 (d, J=8.2 Hz, 1H), 7.81-7.87 (m, 1H), 7.78 (dt, J=1.0, 7.8 Hz, 1H), 7.60-7.70 (m, 1H), 4.93-5.07 (m, 3H), 2.36 (qd, J=6.8, 14.1 Hz, 1H), 2.08-2.25 (m, 2H), 1.30-1.43 (m, 2H), 1.18-1.29 (m, 1H), 1.15 (d, J=6.6 Hz, 3H), 1.07-1.13 (m, 1H), 0.94 (d, J=6.7 Hz, 3H), 0.85 (t, J=7.3 Hz, 3H).

### Example 8

### 1-[(1R)-1-tert-butylpentyl]imidazo[4,5-c]quinolin-4-amine

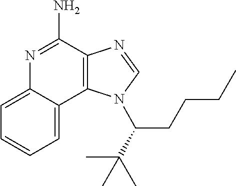 [SMILES: CCCC[C@@H](n1cnc2c(N)nc3ccccc3c21)C(C)(C)C]

### Part A

A 1-L 2-necked round bottom flask, equipped with an addition funnel containing iodine (10.2 g, 40.0 mmol) dissolved in 140 mL of tetrahydrofuran, was charged with L-tert-leucine (5.24 g, 40.0 mmol) and 80 mL of tetrahydrofuran. Sodium borohydride (3.65 g, 96 mmol) was then added to the flask and the mixture was stirred under nitrogen. The iodine solution was then added dropwise over a period of 30 minutes. The reaction mixture was then heated to reflux overnight. The reaction mixture was cooled to ambient temperature and carefully quenched with methanol. The reaction mixture was concentrated under reduced pressure to give a white paste which was dissolved in 70 mL of 20% potassium hydroxide solution. The mixture was extracted with dichloromethane (3×50 mL) and the combined organic layers were dried over Na₂SO₄, filtered and concentrated under reduced pressure to give 4.73 g of (2S)-2-amino-3,3-dimethyl-butan-1-ol as a colorless solid.

### Part B

A solution of (2S)-2-amino-3,3-dimethyl-butan-1-ol (4.73 g, 40.4 mmol) dissolved in 60 mL of dichloromethane was combined with di-tert-butyl dicarbonate (8.81 g, 40.4 mmol) and triethylamine (6.19 mL, 44.5 mmol) and the reaction mixture was stirred overnight. The reaction mixture was then quenched with 10% citric acid solution and the layers were separated. The organic portion was washed successively with 10% citric acid solution, water and brine, dried over Na₂SO₄, filtered and concentrated under reduced pressure to give 5.94 g of tert-butyl N-[(1S)-1-(hydroxymethyl)-2,2-dimethyl-propyl]carbamate as a white solid.

### Part C

A solution of tert-butyl N-[(1S)-1-(hydroxymethyl)-2,2-dimethyl-propyl]carbamate (2.17 g, 10.0 mmol) dissolved in 60 mL of a 1:1 mixture of ethyl acetate/toluene was placed in a round bottom flask. A solution of sodium bromide (1.08 g, 10.5 mmol) dissolved in 5 mL of deionized water was then added to the flask and the mixture was stirred in a −2° C. bath. TEMPO (22 mg) was then added to the stirred mixture followed by the dropwise addition of a solution containing aqueous sodium hypochlorite (4.4% by weight, 18.6 g, 11.0 mmol) and NaHCO₃ (2.56 g, 30 mmol) dissolved in 20 mL of deionized water. After addition was complete, the mixture was stirred for an additional 20 minutes. The mixture was then diluted with ethyl acetate (20 mL) and transferred to a separatory funnel and the layers were separated. The aqueous layer was extracted with an additional 20 mL portion of ethyl acetate. The combined organic portions were successively washed with 30 mL of 10% aqueous citric acid containing 360 mg of potassium iodide, 10% aqueous Na₂S₂O₃, water and finally brine. The organic portion was dried over Na₂SO₄, filtered and concentrated to give 2.15 g of tert-butyl N-[(1S)-1-formyl-2,2-dimethyl-propyl]carbamate as a colorless liquid.

### Part D

A dry 250 mL round bottom flask was charged with propyltriphenylphosphonium bromide (3.05 g, 7.93 mmol) and 30 mL of anhydrous toluene. The reaction mixture was cooled in a 0° C. bath and stirred under a nitrogen atmosphere. An 11% solution of potassium bis(trimethylsilyl)amide in toluene (14.4 g, 7.93 mmol) was then added to the flask. After stirring for 15 minutes the reaction mixture was transferred to a −78° C. bath and a solution of tert-butyl N-[(1S)-1-formyl-2,2-dimethyl-propyl]carbamate (2.15 g, 10.0 mmol) dissolved in 15 mL of anhydrous toluene was added. The stirred mixture was allowed to warm to ambient temperature overnight. The reaction with was quenched by addition of saturated NH₄Cl solution followed by addition of 30 mL of diethyl ether. The layers were separated and the aqueous portion was extracted with an additional 20 mL of diethyl ether. The combined organic layers were washed with brine, dried over MgSO₄, filtered and concentrated under reduced pressure. The resulting material was combined with 25% ethyl acetate/hexanes to precipitate triphenylphosphine oxide which was removed by filtering through a plug of silica gel eluting with 25% ethyl acetate/hexanes. The eluate was concentrated to give a colorless semi-solid. Purification by column chromatography (SiO₂, 7% ethyl acetate/hexanes) gave 1.72 g of tert-butyl N—[(Z,1R)-1-tert-butylpent-2-enyl]carbamate as a white solid.

### Part E

A solution of tert-butyl N—[(Z,1R)-1-tert-butylpent-2-enyl]carbamate (1.72 g) dissolved in 25 mL methanol was placed in a pressure bottle followed by addition of 100 mg of 10% palladium on carbon. The bottle was then shaken under an atmosphere of hydrogen (50 PSI) overnight. The reaction mixture was filtered through a pad of CELITE and the filtrate was concentrated under reduced pressure to give 1.74 g of tert-butyl N-[(1R)-1-tert-butylpentyl]carbamate as a colorless oil.

### Part F

A solution of tert-butyl N-[(1R)-1-tert-butylpentyl]carbamate (1.74 g, 7.15 mmol) dissolved in 20 mL of ethanol was combined with 2 mL of concentrated hydrochloric acid. The stirred reaction mixture was heated to reflux for 90 minutes and then concentrated under reduced pressure to give an oil. Crystallization from acetonitrile gave 602 mg of (3R)-2,2-dimethylheptan-3-amine hydrochloride as white needles.

### Part G

A solution of (3R)-2,2-dimethylheptan-3-amine hydrochloride (602 mg, 3.35 mmol) in 25 mL of methylene chloride was combined with 4-chloro-3-nitroquinoline (696 mg, 3.35 mmol) and triethylamine (1.40 mL, 10.1 mmol) and the reaction mixture was stirred under an atmosphere of nitrogen overnight. The reaction mixture was concentrated to give a yellow solid. The solid was dissolved in 75 mL of ethyl acetate and washed successively with water and brine. The organic portion was dried over Na₂SO₄, filtered and concentrated to give 1.02 g of N-[(1R)-1-tert-butylpentyl]-3-nitro-quinolin-4-amine as a yellow syrup.

### Part H

A solution of N-[(1R)-1-tert-butylpentyl]-3-nitro-quinolin-4-amine (1.02 g, 3.24 mmol) dissolved in 20 mL acetonitrile was placed in a pressure bottle followed by addition of 50 mg of 3% platinum on carbon. The bottle was then shaken under an atmosphere of hydrogen (40 PSI) for 3 hours. The reaction mixture was filtered through a pad of CELITE and the filtrate was concentrated under reduced pressure to give 923 mg of N4-[(1R)-1-tert-butylpentyl]quinoline-3,4-diamine as an orange solid.

### Part I

A solution of N4-[(1R)-1-tert-butylpentyl]quinoline-3,4-diamine (923 mg, 3.24 mmol) dissolved in 25 mL of n-propyl acetate was combined with triethyl orthoformate (1.67 mL, 10.0 mmol) and 50 mg of pyridine hydrochloride and the mixture was heated to 100° C. overnight. The warm reaction mixture was washed successively with saturated NaHCO₃ solution, water and brine. The organic portion was dried over Na₂SO₄, filtered and concentrated to give an orange syrup. Purification by column chromatography (SiO₂, 1-5% methanol/chloroform) gave 740 mg 1-[(1R)-1-tert-butylpentyl]imidazo[4,5-c]quinoline as an orange crystalline solid.

### Part J

A solution of 1-[(1R)-1-tert-butylpentyl]imidazo[4,5-c]quinoline (740 mg, 2.51 mmol) dissolved in 20 mL of methylene chloride was combined with 566 mg of MCPBA (80%) and stirred for 50 minutes. The reaction mixture was combined with 10% Na₂CO₃ solution and 10 mL of methylene chloride the layers were separated. The aqueous portion was further extracted with an additional 10 mL portion of methylene chloride. The combined organic layers were washed with brine and concentrated to give an amber foam. A stirred solution of the amber foam dissolved in 20 mL of methylene chloride was combined with 5 mL of concentrated NH₄OH solution and p-toluenesulfonyl chloride (526 mg, 2.76 mmol). After stirring for 55 minutes, the reaction mixture was diluted with 30 mL of methylene chloride and washed successively with water (3×) and brine. The organic portion was dried over Na₂SO₄, filtered and concentrated under reduced pressure. Purification by column chromatography (SiO₂, 1-7.5% methanol/chloroform) gave a light brown foam. The light brown foam was dissolved in 5 mL of ethanol and 1 mL of concentrated hydrochloric acid. The mixture was evaporated to dryness. Crystallization from acetonitrile gave 271 mg of 1-[(1R)-1-tert-butylpentyl]imidazo[4,5-c]quinolin-4-amine hydrochloride as a cream colored powder.

¹H NMR (500 MHz, METHANOL-d₄) δ 8.60-8.63 (m, 1H), 8.60 (s, 1H), 7.86 (dd, J=1.1, 8.4 Hz, 1H), 7.78 (dt, J=1.1, 7.8 Hz, 1H), 7.66 (ddd, J=1.2, 7.2, 8.3 Hz, 1H), 5.12 (dd, J=3.2, 11.9 Hz, 1H), 2.11-2.33 (m, 2H), 1.28-1.46 (m, 2H), 1.15-1.26 (m, 1H), 1.08 (s, 9H), 0.98-1.06 (m, 1H), 0.83 (t, J=7.4 Hz, 3H).

### Example 9

### (3R)-3-(4-aminoimidazo[4,5-c]quinolin-1-yl)-2-methyl-butan-2-ol

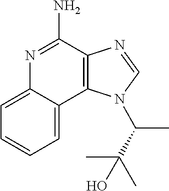 [SMILES: C[C@@H](n1cnc2c(N)nc3ccccc3c21)C(C)(C)O]

### Part A

A suspension of D-alanine methyl ester hydrochloride (4.00 g, 28.2 mmol) in 50 mL of methylene chloride was combined with triethylamine (12.0 mL, 86.4 mmol) and di-tert-butyl dicarbonate. After stirring for 5 hours at ambient temperature, a solution of 5% NaH₂PO₄ was added and the layers were separated. The organic portion was washed successively with saturated NaHCO₃ solution, 10% citric acid solution, water and brine. The organic portion was dried over Na₂SO₄, filtered and concentrated to give 4.83 g of methyl (2R)-2-[(tert-butoxycarbonyl)amino]propanoate as a colorless oil.

### Part B

A stirred solution of methyl (2R)-2-[(tert-butoxycarbonyl)amino]propanoate (2.54 g, 12.5 mmol) dissolved in 200 mL of anhydrous diethyl ether was cooled to −78° C. under an atmosphere of nitrogen. A 3.0 M solution of methyl magnesium bromide in diethyl ether (16.7 mL, 50.0 mmol) was added dropwise over 10 minutes. After addition was complete, the reaction mixture was warmed to 0° C. and stirred for an additional 75 minutes. The reaction mixture was the quenched by careful addition of a saturated solution of NH₄Cl. The layers were separated and the organic portion was washed with water and brine, dried over MgSO₄, filtered and concentrated. Purification by column chromatography (SiO₂, 12% ethyl acetate/hexanes-100% ethyl acetate) gave 1.91 g of tert-butyl N-[(1R)-2-hydroxy-1,2-dimethyl-propyl]carbamate as a colorless oil.

### Part C

To a solution of tert-butyl N-[(1R)-2-hydroxy-1,2-dimethyl-propyl]carbamate (1.91 g, 9.41 mmol) dissolved in 10 mL of ethanol was added 4 mL of concentrated hydrochloric acid. The stirred reaction mixture was heated to reflux for 2 hours and then concentrated under reduced pressure to give a mauve colored solid. Crystallization from acetonitrile gave 1.08 g of (3R)-3-amino-2-methyl-butan-2-ol hydrochloride as colorless needles.

### Part D

A suspension of (3R)-3-amino-2-methyl-butan-2-ol hydrochloride (931 mg, 6.66 mmol) in 20 mL of methylene chloride was combined with 4-chloro-3-nitroquinoline (1.32 g, 6.34 mmoL) and triethylamine (2.65 mL, 19.1 mmol) and the reaction mixture was stirred under an atmosphere of nitrogen overnight. The reaction mixture was concentrated to give a yellow solid. The solid was dissolved in 50 mL of warm ethyl acetate and washed successively with water (2×) and brine. The organic portion was dried over Na₂SO₄, filtered and concentrated to give 1.69 g of (3R)-2-methyl-3-[(3-nitro-4-quinolyl)amino]butan-2-ol as a yellow solid.

### Part E

A solution of (3R)-2-methyl-3-[(3-nitro-4-quinolyl)amino]butan-2-ol (1.69 g, 6.14 mmol) dissolved in 30 mL acetonitrile was placed in a pressure bottle followed by addition of 100 mg of 3% platinum on carbon. The bottle was then shaken under an atmosphere of hydrogen (40 PSI) for 2 hours. The reaction mixture was filtered through a pad of CELITE and the filtrate was concentrated under reduced pressure to give 1.47 g of (3R)-3-[(3-amino-4-quinolyl)amino]-2-methyl-butan-2-ol as a honey colored foam.

### Part F

A solution of (3R)-3-[(3-amino-4-quinolyl)amino]-2-methyl-butan-2-ol (1.47 g, 6.00 mmol) dissolved in 30 mL of n-propyl acetate was combined with triethyl orthoformate (1.49 mL, 9.00 mmol) and 100 mg of pyridine hydrochloride and the mixture was heated to 90° C. overnight. The cooled reaction mixture was diluted with 50 mL of ethyl acetate and washed successively with saturated NaHCO₃ solution, water and brine. The organic portion was dried over Na₂SO₄, filtered and concentrated to give a light brown foam. Purification by column chromatography (SiO₂, 2.4% methanol/chloroform-10% methanol/chloroform) gave 1.15 g of (3R)-3-imidazo[4,5-c]quinolin-1-yl-2-methyl-butan-2-ol as a light tan foam.

### Part G

A solution of (3R)-3-imidazo[4,5-c]quinolin-1-yl-2-methyl-butan-2-ol (1.10 g, 4.31 mmol) dissolved in 25 mL of methylene chloride was combined with 1.30 g of MCPBA (57-86%) and stirred for 40 minutes The reaction mixture was combined with 2% Na₂CO₃ solution and the layers were separated. The aqueous layer was further extracted with several portions of methylene chloride and the combined organic portions were dried over Na₂SO₄, filtered and concentrated to give 0.77 g of a tan solid. The tan solid was suspended in 40 mL of methylene chloride and the mixture was stirred rapidly. Concentrated NH₄OH solution (10 mL) and p-toluenesulfonyl chloride (600 mg, 2.68 mmol) were then added. After stirring for 45 minutes, the reaction mixture was diluted with 25 mL of methylene chloride and washed successively with water (3×) and brine. The organic portion was dried over Na₂SO₄, filtered and concentrated under reduced pressure. Purification by column chromatography (SiO₂, 10% CMA/chloroform-100% CMA) gave a light brown syrup which was crystallized from acetonitrile to give 262 mg of (3R)-3-(4-aminoimidazo[4,5-c]quinolin-1-yl)-2-methyl-butan-2-ol as amber crystals.

¹H NMR (500 MHz, METHANOL-d₄) δ 8.45 (s, 1H), 8.35 (dd, J=0.7, 8.3 Hz, 1H), 7.73 (dd, J=1.0, 8.4 Hz, 1H), 7.52 (ddd, J=1.2, 7.1, 8.3 Hz, 1H), 7.36 (ddd, J=1.3, 7.1, 8.3 Hz, 1H), 5.30 (q, J=7.0 Hz, 1H), 1.76 (d, J=7.0 Hz, 3H), 1.44 (s, 3H), 1.08 (s, 3H).

### Example 10

### (3S)-3-(4-aminoimidazo[4,5-c]quinolin-1-yl)-2-methyl-butan-2-ol

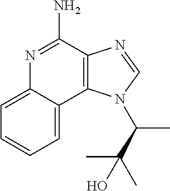 [SMILES: C[C@H](n1cnc2c(N)nc3ccccc3c21)C(C)(C)O]

This compound was prepared from L-alanine methyl ester hydrochloride following the procedures described in Parts A-G for Example 9.

¹H NMR (500 MHz, METHANOL-d₄) δ 8.45 (s, 1H), 8.31-8.38 (m, 1H), 7.73 (dd, J=1.0, 8.4 Hz, 1H), 7.52 (ddd, J=1.2, 7.1, 8.3 Hz, 1H), 7.36 (ddd, J=1.2, 7.1, 8.3 Hz, 1H), 5.31 (q, J=7.0 Hz, 1H), 1.77 (d, J=7.0 Hz, 3H), 1.45 (s, 3H), 1.08 (s, 3H).

### Example 11

### (3R)-3-(4-aminoimidazo[4,5-c]quinolin-1-yl)-2-methyl-pentan-2-ol

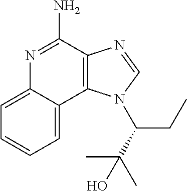 [SMILES: CC[C@@H](n1cnc2c(N)nc3ccccc3c21)C(C)(C)O]

### Part A

A solution of (R)-2-aminobutyric acid (5.00 g, 48.5 mmol) dissolved in 75 mL of anhydrous ethanol was combined with p-toluene sulfonic acid monohydrate (11.3 g, 59.5 mmol) and heated to reflux overnight. The reaction mixture was then concentrated under reduced pressure. The resulting syrup was concentrated from ethanol two more times to give a glassy solid. This was stirred with 150 mL of diethyl ether until a white powder was obtained. The powder was isolated by filtration, rinsed with diethyl ether and dried with suction to give 14.0 g of ethyl (2R)-2-aminobutanoate as the p-toluene sulfonic acid salt.

### Part B

A suspension of (2R)-2-aminobutanoate p-toluene sulfonic acid salt (7.58 g, 25.0 mmol) in 100 mL of methylene chloride was combined with triethylamine (10.4 mL, 75.0 mmol) and di-tert-butyl dicarbonate (6.00 g, 27.5 mmol). After stirring overnight at ambient temperature, the reaction mixture was combined with 5% NaH₂PO₄ solution and the layers were separated. The organic portion was washed successively with saturated NaHCO₃ solution, 10% citric acid solution, water and brine. The organic portion was dried over Na₂SO₄, filtered and concentrated to give 5.40 g of ethyl (2R)-2-(tert-butoxycarbonylamino)butanoate as a colorless oil.

### Part C

A stirred solution of ethyl (2R)-2-(tert-butoxycarbonylamino)butanoate (3.25 g, 14.1 mmol) dissolved in 200 mL of anhydrous diethyl ether was cooled to −78° C. under an atmosphere of nitrogen. A 3.0 M solution of methyl magnesium bromide in diethyl ether (18.7 mL, 56.2 mmol) was added dropwise over 10 minutes. After addition was complete, the reaction mixture was warmed to 0° C. and stirred for an additional 120 minutes. The reaction mixture was then quenched by careful addition of a saturated solution of NH₄Cl. The layers were separated and the organic portion was washed successively with water and brine, dried over MgSO₄, filtered and concentrated. Purification by column chromatography (SiO₂, 10% ethyl acetate/hexanes-33% ethyl acetate/hexanes) gave 1.79 g of tert-butyl N-[(1R)-1-ethyl-2-hydroxy-2-methyl-propyl]carbamate as a colorless oil.

### Part D

A solution of tert-butyl N-[(1R)-1-ethyl-2-hydroxy-2-methyl-propyl]carbamate (1.79 g, 8.25 mmol) dissolved in 15 mL of ethanol was combined with 3 mL of concentrated hydrochloric acid. The stirred reaction mixture was heated to reflux for 2 hours and then concentrated under reduced pressure to give an oil. Repeated concentration from ethanol gave 1.30 of (3R)-3-amino-2-methyl-pentan-2-ol hydrochloride as an amber syrup that solidified on standing.

### Part E

A suspension of (3R)-3-amino-2-methyl-butan-2-ol hydrochloride (1.17 g, 7.61 mmol) in 20 mL of methylene chloride was combined with 4-chloro-3-nitroquinoline (1.42 g, 6.85 mmol) and triethylamine (2.86 mL, 20.6 mmol) and the reaction mixture was stirred under an atmosphere of nitrogen overnight. The reaction mixture was concentrated to give a yellow solid. The solid was dissolved in 50 mL of warm ethyl acetate and washed successively with water (3×) and brine. The organic portion was dried over Na₂SO₄, filtered and concentrated to give a yellow solid. Crystallization from ethyl acetate gave 1.47 g of (3R)-2-methyl-3-[(3-nitro-4-quinolyl)amino]pentan-2-ol as a yellow crystalline solid.

### Part F

A solution of (3R)-2-methyl-3-[(3-nitro-4-quinolyl)amino]pentan-2-ol (1.47 g, 5.09 mmol) dissolved in 25 mL acetonitrile was placed in a pressure bottle followed by addition of 100 mg of 3% platinum on carbon. The bottle was then shaken under an atmosphere of hydrogen (40 PSI) for 2 hours. The reaction mixture was filtered through a pad of CELITE and the filtrate was concentrated under reduced pressure to give 1.32 g of (3R)-3-[(3-amino-4-quinolyl)amino]-2-methyl-pentan-2-ol as an orange solid.

### Part G

A solution of (3R)-3-[(3-amino-4-quinolyl)amino]-2-methyl-pentan-2-ol (1.31 g, 5.06 mmol) dissolved in 30 mL of n-propyl acetate was combined with triethyl orthoformate (1.26 mL, 7.59 mmol) and 100 mg of pyridine hydrochloride and the mixture was heated to 90° C. overnight. An additional 1.0 mL of triethyl orthoformate and 50 mg of pyridine hydrochloride were added to the reaction mixture and heating was continued for another day. The cooled reaction mixture was diluted with 30 mL of ethyl acetate and washed successively with saturated NaHCO₃ solution, water and brine. The organic portion was dried over Na₂SO₄, filtered and concentrated to give a light mauve colored syrup. Purification by column chromatography (SiO₂, 2% methanol/chloroform-20% methanol/chloroform) gave 1.10 g (3R)-3-imidazo[4,5-c]quinolin-1-yl-2-methyl-pentan-2-ol as a crusty white foam.

### Part H

A solution of (3R)-3-imidazo[4,5-c]quinolin-1-yl-2-methyl-pentan-2-ol (1.10 g, 4.09 mmol) dissolved in 25 mL of methylene chloride was combined with 1.23 g of MCPBA (57-86%) and stirred for 60 minutes. The reaction mixture was combined with 2% Na₂CO₃ solution and 25 mL of methylene chloride the layers were separated. The aqueous portion was further extracted with three additional 15 mL portions of methylene chloride and the combined organic layers were dried over Na₂SO₄, filtered and concentrated to give an orange foam. The orange foam was dissolved in 25 mL of methylene chloride and the mixture was stirred rapidly. Concentrated NH₄OH solution (10 mL) and p-toluenesulfonyl chloride (858 mg, 4.50 mmol) were then added. After stirring for 60 minutes, the reaction mixture was diluted with 25 mL of methylene chloride and washed successively with water (3×) and brine. The organic portion was dried over Na₂SO₄, filtered and concentrated under reduced pressure. Purification by column chromatography (SiO₂, 10% CMA/chloroform-100% CMA) gave an amber foam which was crystallized from ethyl acetate and hexanes to give 430 mg of (3R)-3-(4-aminoimidazo[4,5-c]quinolin-1-yl)-2-methyl-pentan-2-ol as off-white crystals.

¹H NMR (500 MHz, METHANOL-d₄) δ 8.42 (s, 1H), 8.39 (dd, J=0.8, 8.4 Hz, 1H), 7.74 (dd, J=1.0, 8.4 Hz, 1H), 7.52 (ddd, J=1.2, 7.1, 8.3 Hz, 1H), 7.36 (ddd, J=1.3, 7.1, 8.3 Hz, 1H), 5.07 (dd, J=3.7, 11.6 Hz, 1H), 2.15-2.35 (m, 2H), 1.46 (s, 3H), 1.07 (s, 3H), 0.78 (t, J=7.3 Hz, 3H).

### Example 12

### (3S)-3-(4-aminoimidazo[4,5-c]quinolin-1-yl)-2-methyl-pentan-2-ol

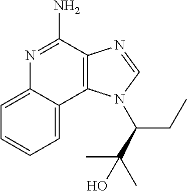 [SMILES: CC[C@H](n1cnc2c(N)nc3ccccc3c21)C(C)(C)O]

This compound was prepared from (S)-2-aminobutyric acid following the procedures described in Parts A-H for Example 11.

¹H NMR (500 MHz, METHANOL-d₄) δ 8.42 (s, 1H), 8.39 (dd, J=1.0, 8.4 Hz, 1H), 7.74 (dd, J=1.0, 8.4 Hz, 1H), 7.53 (ddd, J=1.3, 7.1, 8.3 Hz, 1H), 7.37 (ddd, J=1.3, 7.0, 8.3 Hz, 1H), 5.07 (dd, J=3.7, 11.6 Hz, 1H), 2.16-2.35 (m, 2H), 1.47 (s, 3H), 1.08 (s, 3H), 0.79 (t, J=7.3 Hz, 3H).

### Example 13

### (3R)-3-(4-aminoimidazo[4,5-c]quinolin-1-yl)-2-methyl-hexan-2-ol

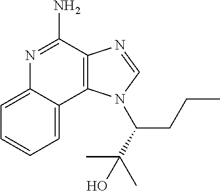 [SMILES: CCC[C@@H](n1cnc2c(N)nc3ccccc3c21)C(C)(C)O]

### Part A

A solution of (R)-2-aminopentanoic acid (5.00 g, 42.7 mmol) dissolved in 75 mL of anhydrous ethanol was treated with p-toluene sulfonic acid monohydrate (9.74 g, 51.3 mmol) and heated to reflux overnight. The reaction mixture was then concentrated under reduced pressure. The resulting syrup was then concentrated from ethanol two more times to give a glassy solid. This was stirred with 150 mL of diethyl ether until a white powder was obtained. The powder was isolated by filtration, rinsed with diethyl ether and dried with suction to give 12.7 g of ethyl (2R)-2-aminopentanoate as the p-toluene sulfonic acid salt.

### Part B

A suspension of ethyl (2R)-2-aminopentanoate p-toluene sulfonic acid salt (7.53 g, 23.8 mmol) in 100 mL of methylene chloride was combined with triethylamine (9.93 mL, 71.4 mmol) and di-tert-butyl dicarbonate (5.70 g, 26.1 mmol). After stirring overnight at ambient temperature, the reaction mixture was combined with 5% NaH₂PO₄ solution and the layers were separated. The organic portion was washed successively with saturated NaHCO₃ solution, 10% citric acid solution, water and brine. The organic portion was dried over Na₂SO₄, filtered and concentrated to give 5.51 g ethyl (2R)-2-(tert-butoxycarbonylamino)pentanoate as a colorless oil.

### Part C

A stirred solution of ethyl (2R)-2-(tert-butoxycarbonylamino)pentanoate (3.75 g, 15.3 mmol) dissolved in 200 mL of anhydrous diethyl ether was cooled to −78° C. under an atmosphere of nitrogen. A 3.0 M solution of methyl magnesium bromide in diethyl ether (20.4 mL, 61.2 mmol) was added dropwise over 10 minutes. After addition was complete, the reaction mixture was warmed to 0° C. and stirred for an additional 120 minutes. The reaction mixture was then quenched by careful addition of a saturated solution of NH₄Cl. The layers were separated and the organic portion was washed successively with water and brine, dried over MgSO₄, filtered and concentrated. Purification by column chromatography (SiO₂, 10% ethyl acetate/hexanes-25% ethyl acetate/hexanes) gave 2.60 g of tert-butyl N-[(1R)-1-(1-hydroxy-1-methyl-ethyl)butyl]carbamate as a colorless syrup.

### Part D

A solution of tert-butyl N-[(1R)-1-(1-hydroxy-1-methyl-ethyl)butyl]carbamate (2.59 g, 11.2 mmol) dissolved in 15 mL of ethanol was combined with 3 mL of concentrated hydrochloric acid. The stirred reaction mixture was heated to reflux for 2 hours and then concentrated under reduced pressure to give an oil. Repeated concentration from ethanol followed by crystallization from acetonitrile gave 1.49 of (3R)-3-amino-2-methyl-pentan-2-ol hydrochloride as colorless needles.

### Part E

A suspension of (3R)-3-amino-2-methyl-pentan-2-ol hydrochloride (1.21 g, 7.21 mmol) in 50 mL of methylene chloride was combined with 4-chloro-3-nitroquinoline (1.34 g, 6.49 mmoL) and triethylamine (2.70 mL, 19.5 mmol) and the reaction mixture was stirred under an atmosphere of nitrogen overnight. The reaction mixture was concentrated to give a yellow solid. The solid was dissolved in 50 mL of ethyl acetate and washed successively with water (3×) and brine. The organic portion was dried over Na₂SO₄, filtered and concentrated to give a yellow solid. Crystallization from ethyl acetate/hexanes gave 1.47 g of (3R)-2-methyl-3-[(3-nitro-4-quinolyl)amino]hexan-2-ol as a yellow crystalline solid.

### Part F

A solution of (3R)-2-methyl-3-[(3-nitro-4-quinolyl)amino]hexan-2-ol (1.47 g, 4.85 mmol) dissolved in 25 mL acetonitrile was placed in a pressure bottle followed by addition of 100 mg of 3% platinum on carbon. The bottle was then shaken under an atmosphere of hydrogen (40 PSI) for 2 hours. The reaction mixture was filtered through a pad of CELITE and the filtrate was concentrated under reduced pressure to give 1.32 g of (3R)-3-[(3-amino-4-quinolyl)amino]-2-methyl-hexan-2-ol as an orange solid.

### Part G

A solution of (3R)-3-[(3-amino-4-quinolyl)amino]-2-methyl-hexan-2-ol (1.31 g, 4.80 mmol) dissolved in 30 mL of n-propyl acetate was combined with triethyl orthoformate (1.20 mL, 7.20 mmol) and 100 mg of pyridine hydrochloride and the mixture was heated to 100° C. overnight. The cooled reaction mixture was diluted with 30 mL of ethyl acetate and washed successively with saturated NaHCO₃ solution, water and brine. The organic portion was dried over Na₂SO₄, filtered and concentrated to give a light mauve colored syrup. Purification by column chromatography (SiO₂, 2% methanol/chloroform-20% methanol/chloroform) gave 1.22 g of (3R)-3-imidazo[4,5-c]quinolin-1-yl-2-methyl-hexan-2-ol as colorless syrup that solidified on standing.

### Part H

A solution of (3R)-3-imidazo[4,5-c]quinolin-1-yl-2-methyl-hexan-2-ol (1.17 g, 4.13 mmol) dissolved in 25 mL of methylene chloride was treated with 1.25 g of MCPBA (57-86%) and stirred for 60 minutes. The reaction mixture was combined with 2% Na₂CO₃ solution and 25 mL of methylene chloride and the layers were separated. The aqueous portion was further extracted with five additional 15 mL portions of methylene chloride. The combined organic layers were dried over Na₂SO₄, filtered and concentrated to give an orange foam. The orange foam was dissolved in 25 mL of methylene chloride and the mixture was stirred rapidly. Concentrated NH₄OH solution (10 mL) and p-toluenesulfonyl chloride (865 mg, 4.54 mmol) were then added. After stirring for 50 minutes, the reaction mixture was diluted with 30 mL of methylene chloride and washed successively with water (3×) and brine. The organic portion was dried over Na₂SO₄, filtered and concentrated under reduced pressure. Purification by column chromatography (SiO₂, 10% CMA/chloroform-100% CMA) gave an amber foam which was crystallized from propyl acetate to give 154 mg of (3R)-3-(4-aminoimidazo[4,5-c]quinolin-1-yl)-2-methyl-hexan-2-ol as golden crystals.

¹H NMR (500 MHz, METHANOL-d₄) δ 8.42 (s, 1H), 8.38 (d, J=8.3 Hz, 1H), 7.74 (d, J=8.3 Hz, 1H), 7.53 (t, J=7.6 Hz, 1H), 7.37 (t, J=7.6 Hz, 1H), 5.16 (dd, J=3.3, 11.9 Hz, 1H), 2.22-2.32 (m, 1H), 2.10-2.20 (m, 1H), 1.46 (s, 3H), 1.11-1.23 (m, 2H), 1.09 (s, 3H), 0.89 (t, J=7.3 Hz, 3H).

### Example 14

### (3S)-3-(4-aminoimidazo[4,5-c]quinolin-1-yl)-2-methyl-hexan-2-ol

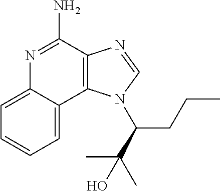 [SMILES: CCC[C@H](n1cnc2c(N)nc3ccccc3c21)C(C)(C)O]

This compound was prepared from (S)-2-aminopentanoic acid following the procedures described in Parts A-H for Example 13.

¹H NMR (500 MHz, METHANOL-d₄) δ 8.42 (s, 1H), 8.38 (dd, J=0.9, 8.4 Hz, 1H), 7.74 (dd, J=1.1, 8.4 Hz, 1H), 7.53 (ddd, J=1.2, 7.1, 8.3 Hz, 1H), 7.37 (ddd, J=1.3, 7.1, 8.3 Hz, 1H), 5.16 (dd, J=3.4, 11.9 Hz, 1H), 2.21-2.32 (m, 1H), 2.20-2.10 (m, 1H), 1.46 (s, 3H), 1.10-1.25 (m, 2H), 1.09 (s, 3H), 0.88 (t, J=7.3 Hz, 3H).

### Example 15

### (3R)-3-(4-aminoimidazo[4,5-c]quinolin-1-yl)-2-methyl-heptan-2-ol

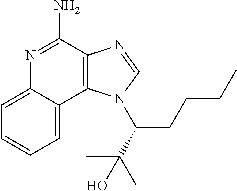 [SMILES: CCCC[C@@H](n1cnc2c(N)nc3ccccc3c21)C(C)(C)O]

### Part A

A 500 mL round bottom flask was charged with 125 mL of anhydrous methanol was cooled to 0° C. followed by the addition of thionyl chloride (3.37 mL, 46.2 mmol). After stirring for 5 minutes, (R)-2-aminohexanoic acid (5.00 g, 38.2 mmol) was added and the reaction mixture was heated to reflux overnight. The reaction mixture was then concentrated under reduced pressure. The resulting syrup was then concentrated from toluene to give an off-white solid. Crystallization from acetonitrile gave 4.96 g of methyl (2R)-2-aminohexanoate hydrochloride as white needles.

### Part B

A suspension of methyl (2R)-2-aminohexanoate hydrochloride (4.96 g, 27.3 mmol) in 150 mL of methylene chloride was cooled to 0° C. and combined with triethylamine (11.4 mL, 81.9 mmol) and di-tert-butyl dicarbonate (5.95 g, 27.3 mmol). The reaction mixture was warmed to ambient temperature and stirring was continued for 5 hours. The reaction mixture was treated with 5% NaH₂PO₄ solution and the layers were separated. The organic portion was washed successively with saturated NaHCO₃ solution, 10% citric acid solution (2×), water and brine. The organic portion was dried over Na₂SO₄, filtered and concentrated to give 6.26 g of methyl (2R)-2-(tert-butoxycarbonylamino)hexanoate as a colorless oil.

### Part C

A stirred solution of methyl (2R)-2-(tert-butoxycarbonylamino)hexanoate (3.24 g, 13.2 mmol) dissolved in 200 mL of anhydrous diethyl ether was cooled to −20° C. under an atmosphere of nitrogen. A 3.0 M solution of methyl magnesium bromide in diethyl ether (17.6 mL, 52.8 mmol) was added dropwise over 10 minutes. After addition was complete, the reaction mixture was warmed to ambient temperature and stirred for an additional 5.5 hours. The reaction mixture was then quenched by careful addition of a saturated solution of NH₄Cl. The layers were separated and the organic portion was washed successively with water and brine, dried over MgSO₄, filtered and concentrated to give 3.21 g of tert-butyl N-[(1R)-1-(1-hydroxy-1-methyl-ethyl)pentyl]carbamate as a colorless syrup which solidified on standing.

### Part D

A solution of tert-butyl N-[(1R)-1-(1-hydroxy-1-methyl-ethyl)pentyl]carbamate (3.21 g, 13.1 mmol) dissolved in 20 mL of ethanol was combined with 4 mL of concentrated hydrochloric acid. The stirred reaction mixture was heated to reflux for 90 minutes and then concentrated under reduced pressure to give an oil. Repeated concentration from acetonitrile gave 2.28 g of (3R)-3-amino-2-methyl-heptan-2-ol hydrochloride as a light purple syrup.

### Part E

A suspension of (3R)-3-amino-2-methyl-heptan-2-ol hydrochloride (2.28 g, 12.2 mmol) in 80 mL of methylene chloride was combined with 4-chloro-3-nitroquinoline (2.28 g, 11.0 mmol) and triethylamine (4.59 mL, 33.0 mmol) and the reaction mixture was stirred under an atmosphere of nitrogen overnight. The reaction mixture was concentrated to give a yellow solid. The solid was dissolved in 75 mL of ethyl acetate and washed successively with water (3×) and brine. The organic portion was dried over Na₂SO₄, filtered and concentrated to give a yellow solid. Crystallization from ethyl acetate/hexanes gave 1.75 g (3R)-2-methyl-3-[(3-nitro-4-quinolyl)amino]heptan-2-ol as a yellow crystalline solid.

### Part F

A solution of (3R)-2-methyl-3-[(3-nitro-4-quinolyl)amino]heptan-2-ol (1.75 g, 5.52 mmol) dissolved in 40 mL acetonitrile was placed in a pressure bottle followed by addition of 100 mg of 3% platinum on carbon. The bottle was then shaken under an atmosphere of hydrogen (40 PSI) for 3 hours. The reaction mixture was filtered through a pad of CELITE and the filtrate was concentrated under reduced pressure to give 1.58 g of (3R)-3-[(3-amino-4-quinolyl)amino]-2-methyl-heptan-2-ol as an orange syrup.

### Part G

A solution of (3R)-3-[(3-amino-4-quinolyl)amino]-2-methyl-heptan-2-ol (1.58 g, 5.52 mmol) dissolved in 50 mL of n-propyl acetate was combined with triethyl orthoformate (2.75 mL, 16.6 mmol) and 75 mg of pyridine hydrochloride and the mixture was heated to 100° C. for 3 days. The cooled reaction mixture was diluted with 25 mL of ethyl acetate and washed successively with saturated NaHCO₃ solution, water and brine. The organic portion was dried over Na₂SO₄, filtered and concentrated to give a light brown syrup. Purification by column chromatography (SiO₂, 1% methanol/chloroform-10% methanol/chloroform) gave 1.36 g (3R)-3-imidazo[4,5-c]quinolin-1-yl-2-methyl-heptan-2-ol as an amber syrup.

### Part H

A solution of (3R)-3-imidazo[4,5-c]quinolin-1-yl-2-methyl-heptan-2-ol (1.36 g, 4.58 mmol) dissolved in 30 mL of methylene chloride was combined with 1.25 g of MCPBA (80%) and stirred for 60 minutes. The reaction mixture was combined with 10% Na₂CO₃ solution and 25 mL of methylene chloride the layers were separated. The aqueous portion was further extracted with an additional 25 mL portion of methylene chloride. The combined organic layers were washed with brine, dried over Na₂SO₄, filtered and concentrated to give an orange syrup. A stirred solution of the orange syrup dissolved in 30 mL of methylene chloride was combined with 10 mL of concentrated NH₄OH solution and p-toluenesulfonyl chloride (960 mg, 5.04 mmol). After stirring for 60 minutes, the reaction mixture was diluted with 30 mL of methylene chloride and washed successively with water (3×) and brine. The organic portion was dried over Na₂SO₄, filtered and concentrated under reduced pressure. Purification by column chromatography (SiO₂, 6% methanol/chloroform saturated with NH₄OH) gave an amber foam. The amber foam was dissolved in 5 mL of ethanol and 1 mL of concentrate hydrochloric acid. The mixture was evaporated to dryness. Crystallization from propyl acetate/2-propanol gave ((3R)-3-(4-aminoimidazo[4,5-c]quinolin-1-yl)-2-methyl-heptan-2-ol hydrochloride as a grey powder.

¹H NMR (500 MHz, METHANOL-d₄) δ 8.58-8.63 (m, 2H), 7.81-7.85 (m, 1H), 7.77 (dt, J=1.0, 7.8 Hz, 1H), 7.64 (dt, J=1.2, 7.78 Hz, 1H), 5.16 (dd, J=3.6, 11.7 Hz, 1H), 2.15-2.34 (m, 2H), 1.47 (s, 3H), 1.26-1.42 (m, 2H), 1.17-1.25 (m, 1H), 1.15 (s, 3H), 0.92-1.06 (m, 1H), 0.83 (t, J=7.4 Hz, 3H).

### Example 16

### (3S)-3-(4-aminoimidazo[4,5-c]quinolin-1-yl)-2-methyl-heptan-2-ol

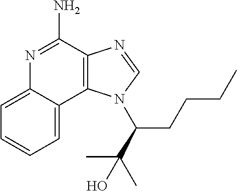 [SMILES: CCCC[C@H](n1cnc2c(N)nc3ccccc3c21)C(C)(C)O]

This compound was prepared from (S)-2-aminohexanoic acid following the procedures described in Parts A-H for Example 15.

¹H NMR (500 MHz, METHANOL-d₄) δ 8.61 (s, 1H), 8.60 (s, 1H), 7.81-7.85 (m, 1H), 7.77 (dt, J=1.0, 7.8 Hz, 1H), 7.61-7.66 (m, 1H), 5.16 (dd, J=3.6, 11.6 Hz, 1H), 2.14-2.34 (m, 2H), 1.47 (s, 3H), 1.26-1.41 (m, 2H), 1.16-1.25 (m, 1H), 1.15 (s, 3H), 0.94-1.05 (m, 1H), 0.83 (t, J=7.4 Hz, 3H).

### Example 17

### (3R)-3-(4-aminoimidazo[4,5-c]quinolin-1-yl)-2,5-dimethyl-hexan-2-ol

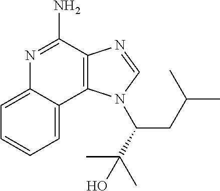 [SMILES: CC(C)C[C@@H](n1cnc2c(N)nc3ccccc3c21)C(C)(C)O]

### Part A

A 1 L round bottom flask was charged with 250 mL of anhydrous methanol was cooled to 0° C. followed by the addition of thionyl chloride (4.00 mL, 54.8 mmol). After stirring for 5 minutes, D-leucine (5.42 g, 41.4 mmol) was added and the reaction mixture was heated to reflux overnight. The reaction mixture was then concentrated under reduced pressure. The resulting syrup was concentrated from toluene to give an off-white solid. Crystallization from acetonitrile gave 6.08 g of methyl D-leucine hydrochloride as white needles.

### Part B

A suspension of methyl D-leucine hydrochloride (6.07 g, 33.4 mmol) in 150 mL of methylene chloride was cooled to 0° C. and combined with triethylamine (13.9 mL, 100 mmol) and di-tert-butyl dicarbonate (7.29 g, 33.4 mmol). The reaction mixture was warmed to ambient temperature and stirring was continued overnight. The reaction mixture was treated with 5% NaH₂PO₄ solution and the layers were separated. The organic portion was washed successively with saturated NaHCO₃ solution, 10% citric acid solution, water and brine. The organic portion was dried over Na₂SO₄, filtered and concentrated to give 7.36 g of methyl (2R)-2-(tert-butoxycarbonylamino)-4-methyl-pentanoate as a colorless oil.

### Part C

A stirred solution of methyl (2R)-2-(tert-butoxycarbonylamino)-4-methyl-pentanoate (4.00 g, 16.3 mmol) dissolved in 200 mL of anhydrous diethyl ether was cooled to −20° C. under an atmosphere of nitrogen. A 3.0 M solution of methyl magnesium bromide in diethyl ether (21.8 mL, 65.3 mmol) was added dropwise over 10 minutes. After addition was complete, the reaction mixture was warmed to ambient temperature and stirred for an additional 5 hours. The reaction mixture was then quenched by careful addition of a saturated solution of NH₄Cl. The layers were separated and the organic portion was washed with water and brine, dried over MgSO4, filtered and concentrated to give 3.75 g tert-butyl N-[(1R)-1-(1-hydroxy-1-methyl-ethyl)-3-methyl-butyl]carbamate as a colorless syrup.

### Part D

A solution of tert-butyl N-[(1R)-1-(1-hydroxy-1-methyl-ethyl)-3-methyl-butyl]carbamate (3.75 g, 15.3 mmol) dissolved in 20 mL of ethanol was combined with 4 mL of concentrated hydrochloric acid. The stirred reaction mixture was heated to reflux for 90 minutes and then concentrated under reduced pressure to give an oil. Crystallization from acetonitrile gave 2.00 g of (3R)-3-amino-2,5-dimethyl-hexan-2-ol hydrochloride as colorless needles. A second crop of crystals (0.36 g) was obtained from the filtrate.

### Part E

A suspension of (3R)-3-amino-2,5-dimethyl-hexan-2-ol hydrochloride (2.28 g, 11.0 mmol) in 80 mL of methylene chloride was combined with 4-chloro-3-nitroquinoline (2.00 g, 11.0 mmol) and triethylamine (4.59 mL, 33.0 mmol) and the reaction mixture was stirred under an atmosphere of nitrogen overnight. The reaction mixture was concentrated to give a yellow solid. The solid was dissolved in 50 mL of ethyl acetate and washed successively with water (3×) and brine. The organic portion was dried over Na₂SO₄, filtered and concentrated to give a yellow syrup. Crystallization from ethyl acetate/hexanes gave 2.10 g of (3R)-2,5-dimethyl-3-[(3-nitro-4-quinolyl)amino]hexan-2-ol as a yellow crystalline solid.

### Part F

A solution of (3R)-2,5-dimethyl-3-[(3-nitro-4-quinolyl)amino]hexan-2-ol (2.10 g, 6.62 mmol) dissolved in 50 mL acetonitrile was placed in a pressure bottle followed by addition of 100 mg of 3% platinum on carbon. The bottle was then shaken under an atmosphere of hydrogen (40 PSI) for 3 hours. The reaction mixture was filtered through a pad of CELITE and the filtrate was concentrated under reduced pressure to give 1.90 g of (3R)-3-[(3-amino-4-quinolyl)amino]-2,5-dimethyl-hexan-2-ol as an orange syrup.

### Part G

A solution of (3R)-3-[(3-amino-4-quinolyl)amino]-2,5-dimethyl-hexan-2-ol (1.90 g, 6.62 mmol) dissolved in 60 mL of n-propyl acetate was combined with triethyl orthoformate (3.30 mL, 19.9 mmol) and 100 mg of pyridine hydrochloride and the mixture was heated to 100° C. overnight. The cooled reaction mixture was washed successively with saturated NaHCO₃ solution, water and brine. The organic portion was dried over Na₂SO₄, filtered and concentrated to give a light brown syrup. Crystallization from acetonitrile gave 1.42 g (3R)-3-imidazo[4,5-c]quinolin-1-yl-2,5-dimethyl-hexan-2-ol as amber crystals.

### Part H

A solution of (3R)-3-imidazo[4,5-c]quinolin-1-yl-2,5-dimethyl-hexan-2-ol (1.42 g, 4.78 mmol) dissolved in 30 mL of methylene chloride was combined with 1.08 g of MCPBA (80%) and stirred for 60 minutes. The reaction mixture was combined with 10% Na₂CO₃ solution and 25 mL of methylene chloride the layers were separated. The aqueous portion was further extracted an additional 25 mL portion of methylene chloride. The combined organic layers were washed with brine, dried over Na₂SO₄, filtered and concentrated to give an orange foam. A stirred solution of the orange foam dissolved in 30 mL of methylene chloride was combined with 10 mL of concentrated NH₄OH solution and p-toluenesulfonyl chloride (1.05 g, 5.52 mmol). After stirring for 60 minutes, the reaction mixture was diluted with 30 mL of methylene chloride and washed successively with water (3×) and brine. The organic portion was dried over Na₂SO₄, filtered and concentrated under reduced pressure. Purification by column chromatography (SiO₂, 6.7% methanol/chloroform saturated with NH₄OH) gave an amber foam. Crystallization from propyl acetate gave 742 mg of (3R)-3-(4-aminoimidazo[4,5-c]quinolin-1-yl)-2,5-dimethyl-hexan-2-ol as off-white crystals.

¹H NMR (500 MHz, METHANOL-d₄) δ 8.36-8.43 (m, 2H), 7.75 (dd, J=1.0, 8.4 Hz, 1H), 7.53 (ddd, J=1.2, 7.1, 8.3 Hz, 1H), 7.38 (ddd, J=1.2, 7.1, 8.3 Hz, 1H), 5.20 (dd, J=3.2, 12.2 Hz, 1H), 2.35 (ddd, J=3.5, 11.9, 14.9 Hz, 1H), 1.88 (ddd, J=3.3, 10.7, 14.4 Hz, 1H), 1.45 (s, 3H), 1.13-1.22 (m, 1H), 1.12 (s, 3H), 0.91 (d, J=6.7 Hz, 3H), 0.85 (d, J=6.6 Hz, 3H).

### Example 18

### (3S)-3-(4-aminoimidazo[4,5-c]quinolin-1-yl)-2,5-dimethyl-hexan-2-ol

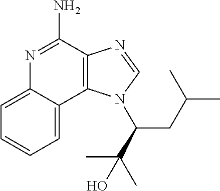 [SMILES: CC(C)C[C@H](n1cnc2c(N)nc3ccccc3c21)C(C)(C)O]

This compound was prepared from L-leucine following the procedures described in Parts A-H for Example 17.

¹H NMR (500 MHz, METHANOL-d₄) δ 8.36-8.44 (m, 2H), 7.75 (dd, J=1.0, 8.4 Hz, 1H), 7.50-7.56 (m, 1H), 7.34-7.42 (m, 1H), 5.19 (dd, J=3.2, 12.1 Hz, 1H), 2.28-2.39 (m, 1H), 1.88 (ddd, J=3.2, 10.7, 14.3 Hz, 1H), 1.45 (s, 3H), 1.16 (m, 1H), 1.12 (s, 3H), 0.90 (d, J=6.7 Hz, 3H), 0.84 (d, J=6.5 Hz, 3H).

### Example 19

### (2R)-2-(4-aminoimidazo[4,5-c]quinolin-1-yl)-3-ethyl-pentan-3-ol

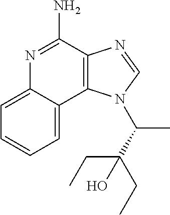 [SMILES: CCC(O)(CC)[C@@H](C)n1cnc2c(N)nc3ccccc3c21]

### Part A

A stirred solution of methyl (2R)-2-[(tert-butoxycarbonyl)amino]propanoate (2.03 g, 10.0 mmol) dissolved in 150 mL of anhydrous diethyl ether was cooled to −40° C. under an atmosphere of nitrogen. A 3.0 M solution of ethyl magnesium bromide in diethyl ether (14 mL, 42 mmol) was added dropwise over 10 minutes. After addition was complete, the reaction mixture was warmed to ambient temperature and stirred for an additional 3.5 hours. The reaction mixture was then quenched by careful addition of a saturated solution of NH₄Cl. The layers were separated and the organic portion was washed successively with water and brine, dried over MgSO₄, filtered and concentrated to give 2.24 g of tert-butyl N-[(1R)-2-ethyl-2-hydroxy-1-methyl-butyl]carbamate as a colorless syrup.

### Part B

A solution of tert-butyl N-[(1R)-2-ethyl-2-hydroxy-1-methyl-butyl]carbamate (2.24 g, 9.67 mmol) dissolved in 30 mL of ethanol was combined with 3 mL of concentrated hydrochloric acid. The stirred reaction mixture was heated to reflux for 2 hours and then concentrated under reduced pressure to give a syrup. The syrup was concentrated from hexanes to give 1.67 g of (2R)-2-amino-3-ethyl-pentan-3-ol hydrochloride as a waxy solid.

### Part C

A suspension of (2R)-2-amino-3-ethyl-pentan-3-ol hydrochloride (1.67 g, 9.70 mmol) in 30 mL of methylene chloride was combined with 4-chloro-3-nitroquinoline (2.02 g, 9.70 mmoL) and triethylamine (4.05 mL, 29.1 mmol) and the reaction mixture was stirred under an atmosphere of nitrogen overnight. The reaction mixture was concentrated to give a yellow solid. The solid was dissolved in 50 mL of warm ethyl acetate and washed successively with water (3×) and brine. The organic portion was dried over Na₂SO₄, filtered and concentrated to give a yellow solid. Purification by column chromatography (SiO₂, 5% ethyl acetate/methylene chloride-20% ethyl acetate/methylene chloride) gave 2.50 g of (2R)-3-ethyl-2-[(3-nitro-4-quinolyl)amino]pentan-3-ol as a yellow solid.

### Part D

A solution of (2R)-3-ethyl-2-[(3-nitro-4-quinolyl)amino]pentan-3-ol (2.50 g, 8.25 mmol) dissolved in 30 mL of acetonitrile was placed in a pressure bottle followed by addition of 100 mg of 3% platinum on carbon. The bottle was then shaken under an atmosphere of hydrogen (40 PSI) for 3 hours. The reaction mixture was filtered through a pad of CELITE and the filtrate was concentrated under reduced pressure to give 2.13 g of (2R)-2-[(3-amino-4-quinolyl)amino]-3-ethyl-pentan-3-ol as an orange colored foam.

### Part E

A solution of (2R)-2-[(3-amino-4-quinolyl)amino]-3-ethyl-pentan-3-ol (2.13 g, 7.80 mmol) dissolved in 30 mL of n-propyl acetate was combined with triethyl orthoformate (1.94 mL, 11.7 mmol) and 100 mg of pyridine hydrochloride and the mixture was heated to 90° C. overnight. The reaction mixture was then treated with an additional 2 mL of triethyl orthoformate and heating was continued for 4 hours. The cooled reaction mixture was diluted with 50 mL of ethyl acetate and washed successively with saturated NaHCO₃ solution, water and brine. The organic portion was dried over Na₂SO₄, filtered and concentrated to give a light brown foam. The foam was triturated with hot ethyl acetate, cooled and filtered to give 1.42 g of (2R)-3-ethyl-2-imidazo[4,5-c]quinolin-1-yl-pentan-3-ol as at an solid.

### Part F

A suspension of (2R)-3-ethyl-2-imidazo[4,5-c]quinolin-1-yl-pentan-3-ol (1.42 g, 5.02 mmol) in 75 mL of methylene chloride was combined with 1.16 g of MCPBA (57-86%) and stirred for 60 minutes. The reaction mixture was combined with 10% Na₂CO₃ solution and the layers were separated. The aqueous portion was further extracted with two 25 mL portions of methylene chloride the combined organic portions were dried over Na₂SO₄, filtered and concentrated to give a tan solid. The tan solid was dissolved in 40 mL of methylene chloride and the mixture was stirred rapidly. Concentrated NH₄OH solution (10 mL) and p-toluenesulfonyl chloride (1.05 g, 5.52 mmol) were then added. After stirring for 60 minutes, the reaction mixture was diluted with 25 mL of methylene chloride and washed successively with water (2×) and brine. The organic portion was dried over Na₂SO₄, filtered and concentrated under reduced pressure. Purification by column chromatography (SiO₂, 30% CMA/chloroform-70% CMA/chloroform) gave 1.03 g of an off-white foam. Crystallization from acetonitrile to give 262 mg of (2R)-2-(4-aminoimidazo[4,5-c]quinolin-1-yl)-3-ethyl-pentan-3-ol as amber crystals.

¹H NMR (500 MHz, METHANOL-d₄) δ 8.52 (s, 1H), 8.26 (dd, J=0.8, 8.4 Hz, 1H), 7.74 (dd, J=1.0, 8.4 Hz, 1H), 7.52 (ddd, J=1.3, 7.1, 8.4 Hz, 1H), 7.35 (ddd, J=1.3, 7.1, 8.3 Hz, 1H), 5.35 (q, J=7.0 Hz, 1H), 1.75-1.87 (m, 2H), 1.72 (d, J=6.85 Hz, 3H), 1.37 (m, 1H), 1.07 (m, 1H), 1.05 (t, J=7.5 Hz, 3H), 0.73 (t, J=7.5 Hz, 3H).

### Example 20

### 1-[(1R)-2-fluoro-1,2-dimethyl-propyl]imidazo[4,5-c]quinolin-4-amine

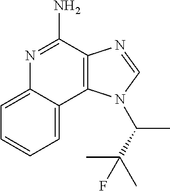 [SMILES: C[C@@H](n1cnc2c(N)nc3ccccc3c21)C(C)(C)F]

### Part A

A solution of tert-butyl N-[(1R)-2-hydroxy-1,2-dimethyl-propyl]carbamate (2.22 g, 10.9 mmol) dissolved in 30 mL of methylene chloride was cooled to −78° C. under a nitrogen atmosphere. Diethylamino sulfur trifluoride (1.73 mL, 13.1 mmol) was then added and the reaction mixture was allowed to warm to ambient temperature overnight. The reaction was then quenched by addition of saturated NaHCO₃ solution and the layers were separated. The organic portion was then washed successively with water and brine. The organic portion was dried over Na₂SO₄, filtered and concentrated to give a light brown syrup. Purification by column chromatography (SiO₂, 10% ethyl acetate/hexanes) gave an orange liquid which was further purified by a second column chromatography ((SiO₂, 12% tert-butyl methyl ether/hexanes) to give 855 mg of tert-butyl N-[(1R)-2-fluoro-1,2-dimethyl-propyl]carbamate as an amber syrup which solidified on standing.

### Part B

To a solution of tert-butyl N-[(1R)-2-fluoro-1,2-dimethyl-propyl]carbamate (855 mg, 4.21 mmol) dissolved in 5 mL of ethanol was added 1 mL of concentrated hydrochloric acid. The stirred reaction mixture was heated to reflux for 90 minutes and then concentrated under reduced pressure to give a white solid. Crystallization from acetonitrile gave 394 mg of (2R)-3-fluoro-3-methyl-butan-2-amine hydrochloride as colorless needles.

### Part C

A solution of 4-chloro-3-nitroquinoline (578 mg, 2.78 mmol) and triethylamine (1.16 mL, 8.34 mmol) dissolved in 30 mL of methylene chloride was combined with (2R)-3-fluoro-3-methyl-butan-2-amine hydrochloride (394 mg, 2.78 mmol) and the reaction mixture was stirred under an atmosphere of nitrogen overnight. The reaction mixture was concentrated to give a yellow solid. The solid was dissolved in 50 mL of warm ethyl acetate and washed successively with water (2×) and brine. The organic portion was dried over Na₂SO₄, filtered and concentrated to give a yellow solid. Crystallization from ethyl acetate gave 483 mg of N-[(1R)-2-fluoro-1,2-dimethyl-propyl]-3-nitro-quinolin-4-amine as yellow crystals. A second crop of crystals (81 mg) was obtained from the filtrate.

### Part D

A solution of N-[(1R)-2-fluoro-1,2-dimethyl-propyl]-3-nitro-quinolin-4-amine (560 mg, 2.02 mmol) dissolved in 10 mL acetonitrile was placed in a pressure bottle followed by addition of 100 mg of 3% platinum on carbon. The bottle was then shaken under an atmosphere of hydrogen (40 PSI) for 3.5 hours. The reaction mixture was filtered through a pad of CELITE and the filtrate was concentrated under reduced pressure to give 500 mg of N4-[(1R)-2-fluoro-1,2-dimethyl-propyl]quinoline-3,4-diamine as an orange solid.

### Part E

A solution of N4-[(1R)-2-fluoro-1,2-dimethyl-propyl]quinoline-3,4-diamine (500 mg, 2.02 mmol) dissolved in 25 mL of n-propyl acetate was combined with triethyl orthoformate (1.00 mL, 6.02 mmol) and 50 mg of pyridine hydrochloride and the mixture was heated to 90° C. overnight. The cooled reaction mixture was diluted with 25 mL of ethyl acetate and washed successively with saturated NaHCO₃ solution, water and brine. The organic portion was dried over Na₂SO₄, filtered and concentrated. Purification by column chromatography (SiO₂, 1% methanol/chloroform-10% methanol/chloroform) gave 456 mg of 1-[(1R)-2-fluoro-1,2-dimethyl-propyl]imidazo[4,5-c]quinoline as a mauve syrup.

### Part F

A solution of 1-[(1R)-2-fluoro-1,2-dimethyl-propyl]imidazo[4,5-c]quinoline (456 mg, 1.77 mmol) dissolved in 20 mL of methylene chloride was combined with 400 mg of MCPBA (80%) and stirred for 90 minutes. The reaction mixture was combined with 10% Na₂CO₃ solution and the layers were separated. The aqueous layer was further extracted with 10 mL of methylene chloride and the combined organic portions were dried over Na₂SO₄, filtered and concentrated to give an orange solid. The orange solid was dissolved in 25 mL of methylene chloride and the mixture was stirred rapidly. Concentrated NH₄OH solution (6 mL) and p-toluenesulfonyl chloride (371 mg, 1.95 mmol) were then added. After stirring for 90 minutes, the reaction mixture was diluted with 25 mL of methylene chloride and washed successively with water (3×) and brine. The organic portion was dried over Na₂SO₄, filtered and concentrated under reduced pressure. Purification by column chromatography (SiO₂, 5% methanol/chloroform saturated with NH₄OH) gave a light brown syrup which was crystallized from acetonitrile to give 188 mg of 1-[(1R)-2-fluoro-1,2-dimethyl-propyl]imidazo[4,5-c]quinolin-4-amine as amber crystals.

¹H NMR (500 MHz, METHANOL-d₄) δ 8.37 (d, J=1.8 Hz, 1H), 8.30 (d, J=8.3 Hz, 1H), 7.73 (dd, J=0.9, 8.4 Hz, 1H), 7.52 (ddd, J=1.2, 7.0, 8.3 Hz, 1H), 7.36 (ddd, J=1.3, 7.0, 8.3 Hz, 1H), 5.50 (dq, J=7.1, 20.1, 1H), 1.83 (d, J=7.1 Hz, 3H), 1.56 (d, J=20.4 Hz, 3H), 1.29 (d, J=20.5 Hz, 3H).

### Example 21

### 1-[(1S)-2-fluoro-1,2-dimethyl-propyl]imidazo[4,5-c]quinolin-4-amine

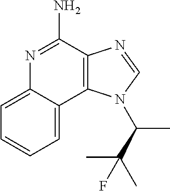 [SMILES: C[C@H](n1cnc2c(N)nc3ccccc3c21)C(C)(C)F]

This compound was prepared from tert-butyl N-[(1S)-2-hydroxy-1,2-dimethyl-propyl]carbamate following the procedures described in Parts A-F for Example 20.

¹H NMR (500 MHz, METHANOL-d₄) δ 8.37 (d, J=2.0 Hz, 1H), 8.29 (d, J=8.3 Hz, 1H), 7.73 (dd, J=1.0, 8.4 Hz, 1H), 7.52 (ddd, J=1.2, 7.1, 8.3 Hz, 1H), 7.35 (ddd, J=1.2, 7.0, 8.3 Hz, 1H), 5.41 (dq, J=7.0, 20.0, 1H), 1.82 (d, J=6.97 Hz, 3H), 1.56 (d, J=20.5 Hz, 3H), 1.29 (d, J=20.5 Hz, 3H).

### Comparative Example 1

### 1-isobutylimidazo[4,5-c]quinolin-4-amine

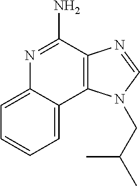 [SMILES: CC(C)Cn1cnc2c(N)nc3ccccc3c21]

Comparative Example 1 (CAS Number 99011-02-6) was prepared as described in U.S. Pat. No. 4,689,338 (Gerster et al.) and in Gerster et al. J. Med. Chem. 2005, 48(10), 3481-3491.

### Comparative Example 2

### 1-(4-aminoimidazo[4,5-c]quinolin-1-yl)-2-methyl-propan-2-ol

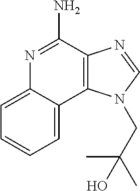 [SMILES: CC(C)(O)Cn1cnc2c(N)nc3ccccc3c21]

Comparative Example 2 (CAS 112668-45-8) was prepared as described in U.S. Pat. No. 4,689,338 (Gerster et al.) and in Gerster et al. J. Med. Chem. 2005, 48(10), 3481-3491.

### Cytokine Induction in Human Cells

Whole blood was obtained from healthy human donors and collected by venipuncture into vacutainer tubes or syringes containing EDTA. Human peripheral blood mononuclear cells (PBMC) were purified from the whole blood by density gradient centrifugation. Histopaque 1077 (15 mL, Sigma, St. Louis, Mo.) was transferred to 6×50 mL sterile polypropylene conical tubes. The Histopaque was overlayed with 15-25 mL of blood diluted 1:2 in Hank's Balanced Salts Solution (HBSS) (Gibco, Life Technologies, Grand Island, N.Y.). The tubes were then centrifuged at 1370 revolutions per minute (rpm) for 30 minutes at 20° C., with no brake (400×g, GH 3.8 A Rotor).

The interface (buffy coat) containing the PBMC was collected and placed in a new sterile 50 mL conical polypropylene centrifuge tube. The PBMC were mixed with an equal volume of HBSS (about 20 mL from the interface and about 20 mL of HBSS), and then centrifuged at 1090 rpm, 10 minutes, 20° C., with brake (270×g, GH 3.8 A Rotor). After completing centrifugation, the cells were resuspended in 2-3 mL ACK Red blood cell lysis buffer (ammonium chloride potassium solution, Gibco, Life Technologies) and incubated for 2-5 minutes at 20° C. Next, HBSS (40 mL) was added to the cells, and the sample was centrifuged at 270×g for 10 minutes at 20° C. The supernatant was decanted, and the cell pellet was resuspended in 5 mL AIM V Medium (Gibco, Life Technologies). Cell aggregates and debris were removed by filtering the cell solution through a BD Falcon 70 micron nylon cell strainer (BD Biosciences, San Jose, Calif.).

The number of viable cells was determined by counting with a Miltenyi FACS instrument (Miltenyi Biotec Inc., San Diego, Calif.) or by using a hemacytometer. For determining cell viability with a hemacytometer, the cells were diluted 1/10 in 0.4% trypan blue and HBSS (specifically, 50 microliter of trypan blue+40 microliter of HBSS+10 microliter of cell solution were added to a microfuge tube and mixed). Ten microliters of the diluted cells were then applied to the hemacytometer, and the number of viable PBMC were determined by microscopy.

The PBMC sample was then resuspended in 96-well plates at a concentration of 8×10⁵ cells/well in 0.1 mL of AIM-V medium. Each compound was solubilized in dimethyl sulfoxide (DMSO) to create a 3 mM stock solution. The stock solution was then further diluted with AIM-V medium to prepare the serial dilutions. The diluted compound (100 microliters) was then transferred to the PBMCs to produce testing sets with final compound concentrations of either 30, 10, 3.3, 1.1, 0.37, 0.12, 0.04, 0.01 micromolar (testing set A); 100, 30, 10, 3.3, 1.1, 0.37, 0.12, 0.04, 0.01, 0.005 micromolar (testing set B); 100, 33.3, 11.1, 3.7, 1.2, 0.41 micromolar (testing set C); or 100, 50, 30, 10, 3.3, 1.1, 0.37, 0.12, 0.04 micromolar (testing set D). The plates also had both positive and negative controls. The negative control wells contained only AIM-V medium with no example compound. The positive control wells contained a control set of imiquimod serially diluted to concentrations of either 30, 10, 3.3, 1.1, 0.37, 0.12, 0.04, 0.01 micromolar (control set A); 100, 30, 10, 3.3, 1.1, 0.37, 0.12, 0.04, 0.01, 0.005 micromolar (control set B); 100, 33.3, 11.1, 3.7, 1.2, 0.41 micromolar (control set C); or 100, 50, 30, 10, 3.3, 1.1, 0.37, 0.12, 0.04 micromolar (control set D). The concentrations used in the control set were selected to match the concentrations used in the testing set. The plates were then cultured at 37° C./5% CO₂ for 21-24 hours. Cell-free supernatants were harvested by centrifuging the 96-well plates at 2100 rpm, 23° C. for 10 minutes. Approximately 160 microliters of the supernatant was then stored in a NUNC 96-well plate, covered with the compression cap and stored at −80° C. until the cytokine analysis was done.

IFN-alpha cytokine levels (picograms/mL) were measured by ELISA (human IFN-alpha, pan specific, Mabtech, Cincinnati, Ohio). IFN-gamma and TNF-alpha levels (picograms/mL) were measured by multiplex bead assay (magnetic beads, R & D Systems, Minneapolis, Minn.) according to the manufacturer's instructions.

The data was analyzed to determine the minimum effective concentration (MEC) for each compound at which induction of a particular cytokine was observed in the assay. Specifically, the minimum effective concentration of each compound (micromolar) was determined as the lowest concentration of the compound that induced a measured cytokine response at a level (pictograms/mL) that was at least 2× greater than that observed with the negative control wells. The results are presented in Table 16a, Table 16b, and Table 16c. The designations “≤0.01,” “≤0.04”, “≤0.4”, “≤0.014” indicate that cytokine induction was observed at the lowest concentration of compound evaluated in the assay (i.e., the lowest of compound in testing sets A, B, C, or D).

TABLE 16a
Cytokine Induction

| | MEC to Induce Cytokine (micromolar) | | |
|---|---|---|---|
| Compound | IFN-alpha | IFN-gamma | TNF-alpha |
| Comparative Example 1 | 11.1 | 11.1 | 11.1 |
| Example 1 | 3.7 | 3.7 | 1.2 |
| Example 2 | >100 | >100 | >100 |
| Example 7 | 0.12 | >30 | 0.12 |

TABLE 16b
Cytokine Induction

| | MEC to Induce Cytokine (micromolar) | | |
|---|---|---|---|
| Compound | IFN-alpha | IFN-gamma | TNF-alpha |
| Comparative Example 2 | 10 | 10 | 10 |
| Example 9 | 3.3 | 3.3 | 10 |
| Example 10 | >30 | >30 | >30 |
| Example 11 | 1.1 | 3.3 | 1.1 |
| Example 12 | >100 | >100 | >100 |
| Example 13 | ≤0.41 | ≤0.41 | ≤0.41 |
| Example 14 | >30 | >30 | >30 |
| Example 15 | ≤0.04 | 0.12 | 0.12 |
| Example 16 | >100 | >100 | >100 |
| Example 17 | 10 | 10 | 10 |
| Example 18 | >100 | >100 | >100 |

TABLE 16c
Cytokine Induction

| | MEC to Induce Cytokine (micromolar) | | |
|---|---|---|---|
| Compound | IFN-alpha | IFN-gamma | TNF-alpha |
| Example 3 | 1.2 | 1.2 | 1.2 |
| Example 4 | >100 | >100 | >100 |
| Example 5 | 0.41 | 1.2 | 0.41 |
| Example 6 | >100 | >100 | >100 |
| Example 8 | 0.04 | >30 | 0.04 |
| Example 19 | 1.1 | 3.3 | 1.1 |
| Example 20 | 10 | 30 | 10 |
| Example 21 | >100 | >100 | >100 |

### TLR Activation and Specificity

HEK-BLUE-hTLR7 or hTLR8 reporter cells were obtained from InvivoGen, San Diego, Calif. According to the manufacturer's description, these reporter cells were prepared by co-transfection of HEK293 cells with an inducible secreted embryonic alkaline phosphatase (SEAP) reporter gene and either the human TLR7 or TLR8 gene. The SEAP reporter gene was placed under the control of an IFN-β minimal promoter fused to five NF-κB and AP-1-binding sites. In the presence of a TLR ligand, activation of NF-κB and AP-1 occurs, resulting in a corresponding increase in SEAP levels.

Parental HEK293 cells (null), which expressed the inducible SEAP reporter, but did not express TLR7 or TLR8, were obtained from InvivoGen and served as the negative control in the assay.

In the assay, the HEK cells were grown and maintained using standard cell culture techniques in a growth medium that contained Dulbecco's Modified Eagle Medium (ThermoFisher Scientific Incorporated, Waltham, Mass.) supplemented with 1% penicillin/streptomycin and 10% heat-inactivated Gibco fetal bovine serum (ThermoFisher Scientific). Each compound was solubilized in DMSO to create a 3 mM stock solution. The stock solution was then further diluted with the growth medium to prepare serial dilutions. Each test compound was tested at a concentration of 30, 10, 3.3, 1.1, 0.37, 0.12, 0.04, and 0.01 micromolar using a 96-well format with 5×10⁴ cells and 200 microliters of growth medium per well.

For each compound, hTLR7, hTLR8, and their respective null control HEK cells were screened. DMSO serially diluted into the growth medium served as the vehicle control. Cell culture supernatants containing the SEAP reporter were collected after an incubation period of 16-20 hours in a cell culture incubator (37° C. and 5% CO₂), and either analyzed immediately or stored at −80° C. SEAP levels were measured using the colorimetric enzyme assay (QUANTI-BLUE (InvivoGen) according to manufacturer's instructions.

The data was analyzed to determine the minimum effective concentration (MEC) for each compound at which activation was observed in the assay. Specifically, the minimum effective concentration of each compound (micromolar) was determined as the lowest concentration of the compound that produced a SEAP expression response at least 2× greater than that observed with the vehicle control wells. The results are presented in Table 17a, Table 17b and Table 17c. The designation “≤0.01” indicates that TLR activation was observed at the lowest concentration of compound evaluated in the assay.

TABLE 17a
TLR Activation

| | MEC to Produce TLR Activation (micromolar) | |
|---|---|---|
| Compound | TLR 7 | TLR 8 |
| Comparative Example 1 | 10 | >30 |
| Example 1 | 1.1 | >30 |
| Example 2 | >30 | >30 |
| Example 7 | 0.04 | >30 |

TABLE 17b
TLR Activation

| | MEC to Produce TLR Activation (micromolar) | |
|---|---|---|
| Compound | TLR 7 | TLR 8 |
| Comparative Example 2 | 10 | >30 |
| Example 9 | 3.3 | >30 |
| Example 10 | >30 | >30 |
| Example 11 | 1.1 | >30 |
| Example 12 | >30 | >30 |
| Example 13 | 0.04 | 10 |
| Example 14 | >30 | >30 |
| Example 15 | 0.01 | 10 |
| Example 16 | >30 | >30 |
| Example 17 | 1.1 | >30 |
| Example 18 | >30 | >30 |

TABLE 17c
TLR Activation

MEC to Produce TLR Activation (micromolar)

| Compound | TLR 7 | TLR 8 |
|---|---|---|
| Example 3 | 1.11 | >30 |
| Example 4 | >30 | >30 |
| Example 5 | 1.11 | >30 |
| Example 6 | >30 | >30 |
| Example 8 | ≤0.01 | >30 |
| Example 19 | 3.3 | >30 |
| Example 20 | 3.3 | >30 |
| Example 21 | >30 | >30 |

The complete disclosures of the patents, patent documents, and publications cited herein are incorporated by reference in their entirety as if each were individually incorporated. Various modifications and alterations to this invention will become apparent to those of ordinary skill in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A compound of Formula (II), or salt thereof: 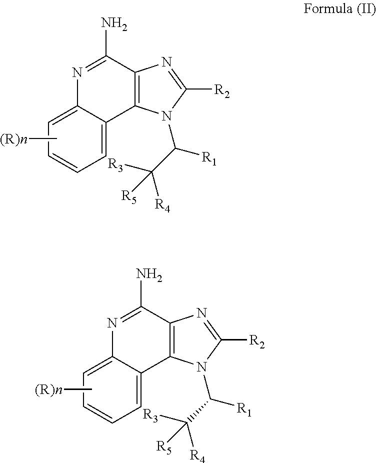
wherein:
n is an integer of 0 or 1;
R is selected from the group consisting of halogen, hydroxy, alkyl, alkoxy, and —C(O)—O-alkyl;
R₁ is a C₁₋₆alkyl;
R₂ is selected from the group consisting of hydrogen, methyl, ethyl, n-propyl, n-butyl, —CH₂OCH₃, —CH₂OCH₂CH₃, and —CH₂CH₂OCH₃;
R₅ is selected from the group consisting of —H, —CH₃, —F, and —OH; and
R₃ is a C₁₋₄alkyl, R₄ is a C₁₋₄alkyl, or R₃ and R₄ are combined to form a ring of 3-7 carbon atoms, optionally having one oxygen atom in the ring, provided that R₅ is not —OH.

2. (canceled)

3. (canceled)

4. The compound or salt of claim 1, wherein R is selected from the group consisting of halogen, hydroxy, —C₁₋₁₂alkyl, —C₁₋₁₂alkoxy, and —C(O)—O—C₁₋₁₀alkyl.

5. The compound or salt of claim 1, wherein n is 0.

6. The compound or salt of claim 1, wherein R₁ is a C₁₋₄alkyl.

7. The compound or salt of claim 1, wherein R₂ is hydrogen.

8. The compound or salt of claim 1, wherein R₃ is a C₁₋₄alkyl and R₄ is a C₁₋₄alkyl.

9. The compound or salt of claim 1, wherein R₁ is selected from the group consisting of —CH₃, —CH₂CH₃, —CH₂CH₂CH₃, —CH₂CH₂CH₂CH₃, and —CH₂CH(CH₃)₂; R₂ is hydrogen; R₃ is methyl or ethyl; R₄ is methyl or ethyl; R₅ is selected from the group consisting of —H, —OH, —CH₃, and —F; and n is 0.

10. The compound or salt of any of claim 1, wherein R₁ is selected from the group consisting of —CH₃, —CH₂CH₃, —CH₂CH₂CH₃, —CH₂CH₂CH₂CH₃, and —CH₂CH(CH₃)₂; R₂ is Hydrogen; R₃ and R₄ are combined to form a ring of 3-7 carbon atoms; R₅ is —H; and n is 0.

11. A pharmaceutical composition comprising an effective amount of a compound or salt of claim 1 in combination with a pharmaceutically acceptable carrier.

12. The pharmaceutical composition of claim 11 further comprising an antigen.

13. (canceled)

14. (canceled)

15. A method of treating a neoplastic disease in a human or animal by administering an effective amount of a compound or salt of claim 1 to the human or animal.

16. The method of claim 15, wherein the treating induces cytokine biosynthesis.
